(19) United States
(12) Patent Application Publication
(10) Pub. No.: US 2018/0250233 A1
(43) Pub. Date: Sep. 6, 2018

(54) TABLETS HAVING MEDIA INDEPENDENT ACTIVE SUBSTANCE DELIVERY

(71) Applicant: MERCK PATENT GMBH, DARMSTADT (DE)

(72) Inventors: Finn BAUER, Bensheim (DE); Thorsten WEDEL, Stockstadt/Rhein (DE); Gudrun BIRK, Darmstadt (DE); Roberto OGNIBENE, Darmstadt (DE); Dieter LUBDA, Bensheim (DE); Guenter MODDELMOG, Reinheim (DE)

(73) Assignee: MERCK PATENT GMBH, DARMSTADT (DE)

(21) Appl. No.: 15/760,097

(22) PCT Filed: Aug. 25, 2016
(86) PCT No.: PCT/EP2016/001431

§ 371 Date: Mar. 14, 2018

(30) Foreign Application Priority Data
Sep. 14, 2015 (EP) 15185029.4
Oct. 9, 2015 (EP) 15189046.4

(51) Int. Cl. A61K 9/20; A61K 31/138

(52) U.S. Cl. CPC A61K 9/2027; A61K 31/138; A61K 9/2077; A61K 9/2054

(57) ABSTRACT

The present invention relates to formulations having extended release of active ingredient, comprising an active ingredient from BCS Class I having high solubility and high permeability in a polyvinyl alcohol-containing matrix, from which the active ingredient is released at a controlled rate over a therapeutically relevant time period independently of the composition of the release medium.

The present invention relates to formulations having extended release of active ingredient, comprising an active ingredient from BCS Class I having high solubility and high permeability in a polyvinyl alcohol-containing matrix, from which the active ingredient is released at a controlled rate over a therapeutically relevant time period independently of the composition of the release medium.

### PRIOR ART

Propranolol belongs to the active ingredient group of beta blockers having antihypertensive, anti-anginal and anti-arrhythmic properties. Although this active ingredient was introduced into therapy as the first δ-receptor blocker as long ago as 1964, and in the meantime a multiplicity of different derivatives in diverse medicament forms are known, especially in order to avoid undesired effects and in order to achieve certain differences in action, propranolol continues to be a frequently administered beta blocker. The substance exhibits good solubility and is absorbed virtually completely after oral administration, but, owing to a pronounced “first-pass” metabolism, has only limited bioavailability of about 25-30%. In addition, the elimination half-life of 2-6 hours is quite short.

Owing to its lipophilicity, propranolol is absorbed virtually completely from the intestine. [Asmar R, Hugues Ch, Pannier B, Daou J, Safar M E; Eur. Heart J. (1987) 8 (Suppl. M):115-120.].

Owing to the good water solubility, conventional administration forms for oral administration of propranolol lead to rapid release of the entire dose of active ingredient in the gastrointestinal tract, meaning that the antihypertensive action commences quickly. At the same time as the short elimination half-life of propranolol, the desired action cannot easily be guaranteed for 12 hours or more. In a conventional formulation, a suitable dose must therefore be administered at least twice daily in order to maintain an adequate concentration of active ingredient in the blood plasma of the patient beyond such a period. However, the necessity for multiple doses distributed over the day easily leads to errors in taking, and to undesired variations in the plasma concentration, which is detrimental to compliance and the therapeutic benefit.

A similar situation also applies to other readily soluble active ingredients having high permeability and having short elimination half lives (active ingredients selected from the substances from BCS Class I), but for which a sustained action throughout the day is desirable. In order to keep the plasma level at an effective concentration level continuously throughout the day, it is therefore necessary to administer a dose a number of times per day.

It is known per se in pharmacology to provide administration forms having extended, or sustained, release of the active ingredients present therein, in order to ensure continuous release of the active ingredient over an extended period.

The prior art discloses extended release formulations for a large number of active ingredients, including β-blockers such as propranolol. The retardation is usually brought about by suitable coatings and/or by embedding the active ingredient in a matrix which controls the release.

In the case of retardation by means of a coating, a core containing the active ingredient is provided with a coating of hydrophilic and/or hydrophobic polymers which delays release of the active ingredient. In the case of retardation by means of a matrix, the active ingredient is embedded in a polymer matrix which controls release of the active ingredient.

The preparation of extended release formulations of this type usually comprises particular process steps, but where appropriate also particular measures, such as the production of a special coating, and where appropriate the use of particularly selected compounds or polymers by means of which delayed release of active ingredient is induced.

Object

Owing to the disadvantageous kinetic properties of propranolol or of other active ingredients or substances from BCS Class I, multiple doses per day are usually necessary, which frequently leads to inadequate patient compliance and consequently an unsatisfactory therapeutic result. The aim is thus to reduce the frequency with which the medication is taken to a single dose per day, for example by administering the active ingredient, such as, for example, propranolol, in the form of a tablet having extended release of active ingredient.

It is therefore also an object of the present invention to provide, in a process which is simple to carry out, extended release formulations from which the release of active ingredient takes place uniformly over several hours, irrespective of the pH of the solution reagent, so that, for example during the release of propranolol, the risk of so-called “dose dumping” can be avoided. It is furthermore an aim of the present invention to suppress dose dumping when medication is taken at the same time as alcoholic drinks.

### BRIEF DESCRIPTION OF THE INVENTION

Experiments have now surprisingly found formulations having extended release of active ingredient, comprising a pharmaceutical active ingredient and polyvinyl alcohols (PVAs) as matrix, where the release of the active ingredient takes place over a therapeutically relevant time period independently of the composition of the release medium. Corresponding formulations have a release of active ingredient which is independent of the pH and ethanol content of the release medium. In particular at a pH in the range from 1 to 7, but also in the case of an alcohol content in the range from 5 to 40% by vol. in the release medium, the formulations according to the invention have an active-ingredient release behaviour which is independent of the type of medium.

Formulations according to the invention comprise a corresponding pharmaceutical active ingredient and polyvinyl alcohols having an average particle size less than 100 μm. Polyvinyl alcohols (PVAs) of the corresponding particle size with microcrystalline celluloses (MCCs) as a combination in a co-mixture are employed here as matrix in the formulations. Particularly suitable are polyvinyl alcohol(s) selected from grades 18-88, 26-88, 40-88, 48-88 and all grades in between in accordance with the requirements of the Ph. Eur., USP or JPE pharmacopoeias, including grade 28-99 in accordance with the requirements of the JPE or Ph. Eur.

The microcrystalline celluloses used therein preferably have average particle sizes less than 150 μm, preferably average particle size in the range from 100 to 140 μm. PVA and microcrystalline celluloses are present in the co-mixtures in the ratio 1:0.5 to 1:2, preferably in the ratio of 1:1, based on the weight. Mixing the co-mixtures with one or more pharmaceutical active ingredient(s), selected from the group of the substances from BCS Class I having high solubility and high permeability, and further processing advantageously gives the formulations according to the invention, which have the desired delayed release of active ingredient at a pH in the range from 1 to 7, but also the alcohol resistance described above. In particular, these properties have been evident in formulations which comprise the active ingredient propranolol and/or pharmaceutically tolerated salts, hydrates or solvates thereof, as antihypertensive β-blocker. This preferably applies to the active ingredient propranolol hydrochloride.

The active-ingredient-containing formulations according to the invention preferably comprise co-mixtures of polyvinyl alcohol (PVA) and microcrystalline celluloses (MCC) in an amount such that the PVA/MCC content in the final tablet is in the range between 1 to 99% by weight, preferably 5 to 95% by weight, in particular in the range from 10 to 90% by weight, based on the total weight of the tablet. Active-ingredient-containing formulations characterised in this way can be obtained using low compression forces and low ejection forces and, as pressed products or compressed tablets, have high tablet hardnesses and low friabilities. In particular, the directly compressible composition employed for the production of the tablets, comprising propranolol hydrochloride as active ingredient and a co-mixture consisting of fine-grained PVA and fine-grained MCC, can be pressed by compression with a compression force of 20 kN to give tablets having hardnesses of greater than/equal to 200 N, which on the other hand have a friability of less than/equal to 0.1% by weight. In a very particularly preferred embodiment, tablets having hardnesses of greater than/equal to 100 N, which on the other hand have a friability of less than/equal to 0.15% by weight, can be obtained by the action of compression with a compression force of 10 kN.

The present invention accordingly also relates to a tablet, produced from a directly compressible composition comprising propranolol hydrochloride and a co-mixture consisting of fine-grained PVA and fine-grained MCC, which has extended release of active ingredient of more than 12 hours, where not more than 22% of the active ingredient originally present in the tablet have been released after one hour, about 25-50% after 3 hours, 50-80% after 6 hours and not less than 80% after 12 hours. A corresponding tablet, having extended release of active ingredient, preferably comprises an active ingredient selected from the group of substances from BCS Class I having high solubility and high permeability, and a co-mixture consisting of fine-grained PVA and fine-grained MCC, where the composition comprises 30-40% by weight of active ingredient, 15-50% by weight of polyvinyl alcohol, 15-50% by weight of microcrystalline cellulose, 0-1% by weight of flow-control agent and 0-1% by weight of lubricant and where the total amount of the ingredients adds up to 100% by weight.

In a particular embodiment, the tablet according to the invention having delayed release of active ingredient comprises propranolol hydrochloride as active ingredient.

In accordance with the invention, the present invention also encompasses a process for the production of the tablets which is simple to carry out, which is characterised in that finely ground PVA, microcrystalline cellulose and the active ingredient are sieved in advance in order to remove coarse particles and are in each case mixed in the desired amount, and with the weighed-out amounts of the other components. The mixture obtained in this way are subsequently pressed or compacted directly to give tablets.

### DETAILED DESCRIPTION OF THE INVENTION

Experiments have shown that the said problems described above in the development of an oral formulation having extended release of active ingredient can, surprisingly, be solved by physically mixing the active ingredient in question with a co-mixture consisting of polyvinyl alcohol (PVA) and microcrystalline cellulose (MCC), adding very small amounts of a flow-control agent and a lubricant, and subsequently converting the mixture into compressed products in a direct compression process in a tableting machine. The co-mixtures of PVAs and microcrystalline cellulose can comprise the two components in the ratio 1:0.5 to 1:2, preferably in the ratio of 1:1, based on the weight. The experiments carried out have now shown that, in particular in the case of the use of propranolol as active ingredient, preferably as the hydrochloride, an advantageous release of propranolol over at least 12 hours can be achieved. Propranolol has in this connection been used as representative as active ingredient from BCS Class I having high solubility and high permeability in a polyvinyl alcohol-containing matrix.

Polyvinyl alcohol (PVA) is a synthetic polymer which is prepared by polymerisation of vinyl acetate and partial hydrolysis of the resultant esterified polymer. Chemical and physical properties of PVA (such as viscosity, solubility, thermal properties, etc.) are highly dependent on its degree of polymerisation (chain length of the PVA polymer) and the degree of hydrolysis. PVA is suitable for a very wide variety of administration forms in the treatment of a multiplicity of diseases. It can therefore be employed in a very wide variety of pharmaceutical dosage forms, including in formulations for ophthalmic, transdermal, topical and in particular for oral applications.

The experiments carried out here have shown, in particular, that the particularly advantageous, delayed release of active ingredient of substances from BCS Class I can be achieved from tabletted formulations in which the polyvinyl alcohols are selected from the group of grades 18-88, 26-88, 40-88, 48-88 and all grades in between in accordance with the requirements of the Ph. Eur., USP or JPE pharmacopoeias, including grade 28-99 in accordance with the requirements of the JPE or Ph. Eur, where the first number of the grade designation refers to the viscosity which arises in aqueous solution at 20° C. as a relative measure of the molecular weight of the polyvinyl alcohol (measured in a 4% solution at 20° C. in accordance with DIN 53 015 in distilled water at a pH in the range 4.5-7 both for partially and also fully hydrolysed polymer, in accordance with DIN 19 260/61). The second number of the grade designation relates to the degree of hydrolysis (degree of saponification) of the parent polyvinyl acetate. The co-mixtures used in accordance with the invention can be prepared using all commercially available polyvinyl alcohols that meet these criteria. The co-mixtures of polyvinyl alcohols (PVAs) and microcrystalline celluloses are prepared using, in particular, PVAs having an average particle size of less than 100 μm.

The experiments described below were carried out with various polyvinyl alcohol grades characterised above, which are available with various article numbers from Merck KGaA, Darmstadt, Germany, for use as excipient (EMPROVE® exp Ph. Eur., USP, JPE).

The second component of the co-mixtures used in accordance with the invention is microcrystalline cellulose (MCC) for pharmaceutical applications and is likewise characterised in the pharmacopoeias. It is obtained by the action of mineral acids from a pulp of plant fibres (cellulose) [Ph. Eur. 2001] [USP 2002] [JP 2001], with α-cellulose, which has degrees of polymerisation of greater than 2000, subsequently being precipitated out of the purified solution with the aid of sodium hydroxide solution. The product obtained is subjected to partial, acidic hydrolysis. The hydrolysis causes depolymerisation, as a result of which the degree of polymerisation of the cellulose fibres drops and the crystalline content increases, since amorphous regions in particular are removed. Subsequent drying, for example spray drying or drying in a stream of air, gives the pulverulent, free-flowing products of the MCC of various particle size.

MCC is used in broad areas of the pharmaceutical industry. It is employed as filler for capsules and tablets, dry binder, disintegration promoter or disintegrant, gel former and as addition to tablet-coating suspensions.

In order to carry out the present invention, MCC which is commercially available from JRS Pharma (Rosenberg, Germany) under the trade name Vivapur® Type 102 is used in the co-mixtures. This microcrystalline cellulose has per se an average particle size of 100 μm and a water content of less than 7%. In addition, comparable MCC grades which can be employed in the same way are commercially available under other product names. In general, pharmaceutical grade microcrystalline celluloses having an average particle size of less than 150 μm are suitable for the preparation of the co-mixtures according to the invention. Preference is given to the use of microcrystalline celluloses which have average particle sizes in the range from 100 to 140 μm.

A detailed list of the particle size distribution of the MCC used here is given below in the “Characterisation of the raw materials used” section. This MCC has very good flowability and is tabletable. In the co-mixtures described here, the addition of MCC supports both tabletability of the formulation and also delayed release of active ingredient from the tablet in the application.

The use of the hydrophilic polymer polyvinyl alcohol (PVA) in combination with microcrystalline cellulose results in swelling of the tablet and gel formation in the presence of liquid from the gastrointestinal system or also in slowed erosion of the tablet in the course of the residence time in the gastrointestinal tract. This has the consequence that delayed release of active ingredient from the PVA matrix occurs.

The formulations according to the invention, which are prepared using co-mixtures of PVA and MCC with the more precisely specified grades and mixing ratios below, are distinguished by the fact that they
1. are very simple and thus inexpensive, and substantially complication-free, to prepare,
2. surprisingly exhibit pH independence of the in-vitro release of propranolol in the pH range from 1 to 7, where propranolol hydrochloride has been used as representative as active ingredient from BCS Class I having high solubility and high permeability, and
3. advantageously have a release of active ingredient which is virtually uninfluenced by ethanol, where the ethanol concentration in the medium can be up to 40% by vol., preferably 5 to 40% by vol.

In summary, it is thus possible, by means of a simple direct compression process, to obtain a tablet whose release of active ingredient takes place substantially independently of the pH values in the release medium. Furthermore, modified, in particular accelerated, release of active ingredient in alcoholic test media is not evident either. These two properties are an essential prerequisite for preventing any dose dumping effects, i.e. unintended and sudden release of excessive amounts of active ingredient from the administration form during passage through the gastrointestinal tract. The two effects support product safety and thus increase the safety for the patient.

Accordingly, the co-mixtures according to the invention are particularly suitable for the preparation of active-ingredient-containing formulations with substances from BCS Class I. These active ingredients have high solubility and at the same time high permeability. It is assumed that the absorption rate of these active ingredients is determined principally by the rate of gastric emptying. An active ingredient is assigned to BCS Class I if the highest dose of this medicament dissolves completely in a maximum of 250 ml of an aqueous dissolution medium having a pH in the range between 1 and 7.5 and at the same time it has high permeability. The permeability of a medicament is high if at least 90% of an administered dose are absorbed by the body in a certain time. This must be demonstrated by suitable data (for example from mass balance studies).

The present invention enables the pharmaceutical formulation scientist, in a very simple process, to achieve safety-relevant product properties for a tablet formulation having extended release of active ingredient by simple mixing of a predetermined amount of an active ingredient (API) with a PVA/MCC pre-mixture. For this purpose, it is possible to employ PVA/MCC pre-mixtures (co-mixtures) in which PVA and microcrystalline cellulose, each in pharmaceutical grade and having average particle sizes as described above, in the ratio 1:0.5 to 1:2, based on the weight, have been mixed intensively with one another. Preference is given to the use of co-mixtures in which the weight ratio of the two components is 1:1.

In particular, these pre-mixtures or co-mixtures have proven suitable for providing active ingredients from BCS Class I in the form of tablets which enable extended release of this active ingredient. However, these co-mixtures according to the invention can also be used to incorporate active ingredients from other BCS classes, in particular BCS Class II, into a PVA/MCC matrix of this type and to compress them to give tablets.

The active ingredients from BCS Class I include, for example, amiloride, chloroquine, cyclophosphamide, diazepam, doxycycline, metoprolol, metronidazole, phenobarbital, prednisolone, primaquine, propranolol, salicylic acid, theophylline or zidovudine, besides other active ingredients.

In accordance with the invention, the co-mixtures of polyvinyl alcohol (PVA) and microcrystalline cellulose (MCC) described can be used to prepare tablet formulations having delayed release of active ingredient in which the co-mixtures are present in the final tablet in an amount of 1 to 99% by weight, preferably in an amount of 5 to 95% by weight. Particular preference is given to formulations having a content of co-mixture in the range from 10 to 90% weight, based on the total weight of the tablet. By using the co-mixtures described in the tablet formulations, it is possible to produce active-ingredient-containing pressed products or compressed tablets using low compression forces and low injection forces. In this way, tablets having high tablet harnesses and low friabilities are obtained, i.e. tablets having hardnesses greater than/equal to 200 N, which on the other hand have a friability of less than/equal to 0.1% weight, are obtained using compression with a compression force of 20 kN.

Friability here is taken to mean the abrasion that occurs in the case of solid bodies, here in the case of tablets, owing to the action of mechanical energy, for example during transport, storage, but also during further processing or packaging. The friability is determined by standardised methods. The determinations carried out in the examples described here used a TA420 friability tester (Erweka, Germany), by means of which the measurements are carried out in accordance with Ph. Eur. 7th Edition “Friability of Uncoated Tablets”. The instrument works with a fixed speed of rotation of 25 min⁻¹ of the test chamber loaded with tablets. The measurements are in each case carried out one day after tablet production.

The tablet hardness on the other hand relates to the force necessary to crush a compressed tablet comprising the co-mixture between two parallel plates or jaws. The tablet hardness can be measured by producing, in a first step, a tablet by compression of a certain amount of the mixture in a tablet press with a pre-determined compression force. A ram in the compression mould of the tablet press acts on the weighed-out, introduced amount of the mixture with a compression force of, for example, approximately 20 kN. The hardness of the tablet obtained in this way can then be determined by measuring the force necessary to crush the tablet, for example using an Erweka Multicheck® 5.1 tablet hardness tester (Erweka, Germany). The determination of the tablet hardness is described below.

The use of the above-described co-mixtures of fine-grained PVA and fine-grained MCC thus enables the production of a tablet with propranolol hydrochloride as active ingredient which has a release of active ingredient of more than 12 hours, where not more than 22% of the active ingredient have been released after one hour, about 25-50% after 3 hours, 50-80% after 6 hours and not less than 80% after 12 hours. In this case, propranolol hydrochloride serves only as model active ingredient. Comparable results can be achieved with other active ingredients from BCS Class I, since the release of the active ingredient is determined primarily by the properties of the compressed tablet matrix comprising PVA and MCC. For the production of the desired tablets, the mixture can be provided with further assistants which are compatible with the mixture, such as flow-control agents or lubricants. Lubricants which can be employed are all lubricants known for this purpose to the person skilled in the art, so long as they are compatible with the co-mixture according to the invention and the active ingredient used, such as, for example, magnesium stearate, talc, or polyethylene glycols as glidant and lubricant. The same applies to the addition of flow-control agents and other additives.

In accordance with the invention, the present invention accordingly relates to tablets having extended release of active ingredient which comprise an active ingredient selected from the group of the substances from BCS Class I having high solubility and high permeability and a co-mixture of fine-grained PVA and fine-grained MCC, and where the composition comprises 30 to 40% by weight of active ingredient, 15 to 50% by weight of polyvinyl alcohol, 15-50% by weight of microcrystalline cellulose, and optionally tableting assistants. For example, 0 to 1% by weight of flow-control agent and 0 to 1% weight of lubricant may be present therein. In total, the total amount of the ingredients adds up to in each case 100% by weight.

A tablet of this type may comprise, for example, propranolol hydrochloride as active ingredient from BCS Class I.

For the production of the tablets according to the invention, finely ground PVA of the selected grade, as described above, and microcrystalline cellulose are mixed with one another in a predetermined ratio, where the two components have been sieved before mixing in order to remove coarse particles. This mixture is mixed with the active ingredient, which has likewise been pre-sieved, in amounts which have in each case been weighed out with one another. If necessary, tableting assistants are added to the mixture obtained in this way, which is subsequently compressed or compacted directly using suitable devices to give tablets.

The examples given below disclose methods and conditions for the preparation of the active-ingredient-containing extended release formulations according to the invention. It is self-evident to the person skilled in the art that methods for the preparation of the pre-mixtures and the tablet matrices other than those described here are also available.

The examples show the particular advantages of these PVA/MCC combinations.

The present description enables the person skilled in the art to apply the invention comprehensively. Even without further comments, it is therefore assumed that a person skilled in the art will be able to utilise the above description in the broadest scope.

If anything is unclear, it goes without saying that the publications and patent literature cited should be consulted. Accordingly, these documents are regarded as part of the disclosure of the present description.

For better understanding and illustration of the invention, examples are given below which are within the scope of protection of the present invention. These examples also serve to illustrate possible variants. Owing to the general validity of the inventive principle described, however, the examples are not suitable for reducing the scope of protection of the present application to these alone.

Furthermore, it goes without saying for the person skilled in the art that, both in the examples given and also in the remainder of the description, the component amounts present in the compositions always only add up to 100% by weight or mol-%, based on the composition as a whole, and cannot exceed this, even if higher values could arise from the percent ranges indicated. Unless indicated otherwise, % data are thus regarded as % by weight or mol-%, with the exception of ratios, which are reproduced in volume figures.

The temperatures given in the examples and the description as well as in the claims are in ° C.

### EXAMPLES

The conditions for production and for analytical and pharmaceutical formulation testing are evident from the examples. Propranolol extended release tablets are produced in a direct compression process. By way of example, the use of co-mixtures of ground PVA 26-88 or PVA 40-88 with the MCC Vivapur® 102 (JRS) in the ratio 1:1 as retardation matrices is described. The in-vitro release profiles over 12 hours are recorded from the following media: HCl 0.1 M; HCl buffer pH 1.2; phosphate buffer pH 6.8; pH change method: 2 hours HCl 0.1 M and subsequently in phosphate buffer pH 6.8, and media comprising HCl 0.1 M with in each case 5%, 20% and 40% of ethanol (in each case % by vol.).

Instruments/Methods for the Characterisation of the Material Properties
1. Bulk density: in accordance with DIN EN ISO 60: 1999 (German version)
   quoted in “g/ml”
2. Tapped density: in accordance with DIN EN ISO 787-11: 1995 (German version)
   quoted in “g/ml”
3. Angle of repose: in accordance with DIN ISO 4324: 1983 (German version)
   quoted in “degrees”
4. Surface area determined by the BET method: evaluation and procedure in accordance with the literature “BET Surface Area by Nitrogen Absorption” by S. Brunauer et al. (Journal of American Chemical Society, 60, 9, 1983). Instrument: ASAP 2420 Micromeritics Instrument Corporation (USA); nitrogen; sample weight: about 3.0000 g; heating: 50° C. (5 h); heating rate 3 K/min; arithmetic mean from three determinations quoted
5. Particle size determination by laser diffraction with dry dispersal: Mastersizer 2000 with Scirocco 2000 dispersion unit (Malvern Instruments Ltd., UK), determinations at a counterpressure of 1, 2 and 3 bar; Fraunhofer evaluation; dispersant RI: 1.000, obscuration limits: 0.1-10.0%, tray type: general purpose, background time: 7500 msec, measurement time: 7500 msec, procedure in accordance with ISO 13320-1 and the information in the technical manual and specifications from the instrument manufacturer; quoted in % by vol.
6. The tableting tests are carried out as follows:
   The mixtures in accordance with the compositions indicated in the experimental part are mixed for 5 minutes in a sealed stainless-steel container (capacity: about 2 l, height: about 19.5 cm, diameter: about 12 cm outside dimension) in a laboratory tumble mixer (Turbula T2A, Willy A. Bachofen, Switzerland).
   The magnesium stearate employed is Parteck® LUB MST (vegetable magnesium stearate) EMPROVE® exp Ph. Eur., BP, JP, NF, FCC Article No. 1.00663 (Merck KGaA, Germany) which has been passed through a 250 μm sieve.
   The compression to give 500 mg tablets (11 mm punch, round, flat, with bevel edge) is carried out in a Korsch EK 0-DMS instrumented eccentric tableting machine (Korsch, Germany) with the Catman® 5.0 evaluation system (Hottinger Baldwin Messtechnik—HBM, Germany).
   Depending on the compression force tested (nominal settings: ˜5, ˜10, ˜20 and ˜30 kN; the effectively measured actual values are indicated in the examples), at least 100 tablets are produced for evaluation of the compression data and determination of the pharmaceutical characteristics.
   Tablet hardnesses, diameters and heights: Erweka Multicheck® 5.1 (Erweka, Germany); average data (arithmetic means) from in each case 20 tablet measurements per compression force. The measurements are carried out one day after tablet production.
   Tablet abrasion: TA420 friability tester (Erweka, Germany); instrument parameters and performance of the measurements in accordance with Ph. Eur. 7th Edition “Friability of Uncoated Tablets”. The measurements are carried out one day after tablet production.
   Tablet weight: Average (arithmetic mean) from the weighing of 20 tablets per compression force: Multicheck® 5.1 (Erweka, Germany) with Sartorius CPA 64 balance (Sartorius, Germany). The measurements are carried out one day after tablet production.
7. Propanolol release testing: The compressed tablets containing propranolol HCl (compressed with a compression force of 10, 20 or 30 kN) are measured in an in vitro release apparatus from ERWEKA (Heusenstamm, Germany) using the “Apparatus 2 (Paddle Apparatus)” described in Ph. Eur. 8.4 under 2.9.3. “Dissolution test for solid dosage forms” and under the conditions described therein (Ph. Eur.=European Pharmacopoeia). The sampling is carried out automatically via a hose pump system with subsequent measurement in a Lambda® 35 photometer (Perkin Elmer, USA) and a flow cell.
8. Measurement apparatuses and measurement parameters:
   ERWEKA DT70 release apparatus fitted with Apparatus 2 (Paddle Apparatus in accordance with Ph. Eur.)
   Temperature: 37° C.+/−0.5° C.
   Speed of rotation of the paddle: 50 rpm
   Release medium: 900 ml
   (except for pH change method: here media volumes in accordance with Ph. Eur. Method A)
   Total running time of the measurements: 12 hours (with sampling after 15, 30, 45, 60 minutes, subsequently every 60 minutes until the expiry of a total running time of 12 hours (in the tables and graphs here, the data for the 15, 30 and 45 minute samples are not shown)
   Hose pump with sampling: Ismatec IPC, model ISM 931; App. No. 12369-00031
   Lambda® 35 photometer, Perkin Elmer
   Measurement at 214 nm in a 0.5 mm flow measurement cell
   Evaluation via Dissolution Lab Software Version 1.1, Perkin Elmer Inc. (USA)

Release Media Used:
0.1N HCl, Art. No. 109060 (Merck KGaA, Germany)
HCl buffer pH 1.2 acc. to Ph. Eur.
phosphate buffer pH 6.8 acc. to Ph. Eur.
pH change method: 2 hours 0.1N HCl, then re-buffering to pH 6.8 as described in PH. Eur. 8.4 under 2.9.3. for method A
ethanol 40% by vol. (% v/v): mixture consisting of 6 parts by volume of 0.1N HCl, Art. No. 109060 (Merck KGaA, Germany) and 4 parts by volume of absolute ethanol, Art. No. 100983 (Merck KGaA, Germany)
ethanol 20% by vol. (% v/v): mixture consisting of 8 parts by volume of 0.1N HCl, Art. No. 109060 (Merck KGaA, Germany) and 2 parts by volume of absolute ethanol, Art. No. 100983 (Merck KGaA, Germany)
ethanol 5% by vol. (% v/v): mixture consisting of 9.5 parts by volume of 0.1N HCl, Art. No. 109060 (Merck KGaA, Germany) and 0.5 parts by volume of absolute ethanol, Art. No. 100983 (Merck KGaA, Germany)

Characterisation of the Raw Materials Used

1. PVA 40-88 and PVA 26-88
1.1 Raw materials for grinding
1.1.1. PVA 26-88: polyvinyl alcohol 26-88, suitable for use as excipient EMPROVE® exp Ph. Eur., USP, JPE, Article No. 1.41352, Merck KGaA, Darmstadt, Germany
1.1.2. PVA 40-88: polyvinyl alcohol 40-88, suitable for use as excipient EMPROVE® exp Ph. Eur., USP, JPE, Article No. 1.41353, Merck KGaA, Darmstadt, Germany

These PVA grades are in the form of coarse particles with a size of several millimetres, which cannot be employed in this form as a directly compressible tableting matrix.

The coarse particles do not allow reproducible filling of the dies and thus do not enable a constant tablet weight to be achieved, even at high rotational speeds of the (rotary) tableting machines. In addition, only fine-grained PVAs are able to ensure homogeneous distribution of the active ingredient. in the tablet without the occurrence of separation effects. However, this is vital for ensuring individual dosage accuracy of the active ingredient. (content uniformity) in each tablet produced. In addition, only a fine-grained PVA can ensure the homogeneous gel formation throughout the tablet body that is necessary for reproducible retardation.

For these reasons, the above-mentioned coarse-grained PVA grades must be comminuted, i.e. ground, before use as directly compressible retardation matrices.
1.2 Ground PVA grades
1.2.1. Ground PVA 26-88, from polyvinyl alcohol 26-88 (Article No. 1.41352) having the average particle-size fractions Dv50 (laser diffraction; dry dispersal):
   Dv50 84.88-87.60 μm
1.2.2. Ground PVA 40-88, from polyvinyl alcohol 40-88 (Article No. 1.41353) having the average particle-size fractions Dv50 (laser diffraction; dry dispersal):
   Dv50 85.84-87.37 μm

Grinding:

The grinding of the PVA grades is carried out in an Aeroplex® 200 AS spiral jet mill from Hosokawa Alpine, Augsburg, Germany, under liquid nitrogen as cold grinding at temperatures in the range from 0° C. to minus 30° C. The desired particle size is produced empirically, in particular by variation of the grinding temperature, i.e. the grinding conditions are varied by ongoing in-process controls of the particle size until the desired particle size fraction is obtained.

The resultant product properties of the ground PVA grades, in particular the powder characteristics, such as bulk density, tapped density, angle of repose, BET surface area, BET pore volume as well as the particle size distributions, are evident from the following tables:

Bulk Density, Tapped Density, Angle of Repose, BET Surface Area, BET Pore Volume:

(Details on the Measurement Methods, See Under Methods)

| | | | | BET | BET |
|---|---|---|---|---|---|
| | Bulk | Tapped | Angle of | surface | pore |
| | density | density | repose | area | volume |
| Sample | (g/ml) | (g/ml) | (°) | (m²/g) | (cm³/g) |
| PVA 26-88* | 0.51 | 0.70 | 36.7 | 0.35 | 0.0019 |
| PVA 40-88* | 0.51 | 0.70 | 34.0 | 0.33 | 0.0018 |

*ground PVA

Particle Distribution Determined by Laser Diffraction with Dry Dispersal (1 bar Counterpressure): Figures in μm (Details on the Measurement Method, See Under Methods

| Sample | Dv5 | Dv10 | Dv20 | Dv25 | Dv30 | Dv50 | Dv75 | Dv90 | Dv95 |
|---|---|---|---|---|---|---|---|---|---|
| PVA 26-88* | 17.39 | 24.78 | 38.52 | 45.59 | 52.97 | 87.60 | 161.70 | 285.80 | 526.73 |
| PVA 40-88* | 16.33 | 23.54 | 37.10 | 44.13 | 51.49 | 85.96 | 156.09 | 245.33 | 304.05 |

*ground PVA

Particle Distribution Determined by Laser Diffraction with Dry Dispersal (2 bar Counterpressure):

Figures in μm (Details on the Measurement Method, See Under Methods)

| Sample | Dv5 | Dv10 | Dv20 | Dv25 | Dv30 | Dv50 | Dv75 | Dv90 | Dv95 |
|---|---|---|---|---|---|---|---|---|---|
| PVA 26-88* | 16.15 | 23.53 | 37.22 | 44.26 | 51.56 | 85.05 | 151.30 | 240.02 | 305.79 |
| PVA 40-88* | 15.46 | 22.54 | 36.12 | 43.27 | 50.77 | 85.84 | 156.51 | 247.86 | 309.84 |

*ground PVA

Particle Distribution Determined by Laser Diffraction with Dry Dispersal (3 bar Counterpressure):

Figures in μm (Details on the Measurement Method, See Under Methods)

| Sample | Dv5 | Dv10 | Dv20 | Dv25 | Dv30 | Dv50 | Dv75 | Dv90 | Dv95 |
|---|---|---|---|---|---|---|---|---|---|
| PVA 26-88* | 15.99 | 23.44 | 37.29 | 44.35 | 51.56 | 84.88 | 150.53 | 237.38 | 299.34 |
| PVA 40-88* | 15.50 | 22.86 | 36.99 | 44.35 | 52.00 | 87.37 | 158.92 | 250.34 | 310.78 |

*ground PVA

2. Microcrystalline Celluloses (MCCs)

Vivapur® Type 102 Premium, microcrystalline cellulose, Ph. Eur., NF, JP, JRS Pharma, Rosenberg, Germany

Particle Distribution Determined by Laser Diffraction with Dry Dispersal (1 bar Counterpressure):

Figures in μm (Details on the Measurement Method, See Under Methods)

| Sample | Dv10 | Dv20 | Dv25 | Dv30 | Dv50 | Dv75 | Dv90 |
|---|---|---|---|---|---|---|---|
| Vivapur ® 102 | 31.56 | 53.04 | 66.00 | 79.89 | 135.87 | 215.53 | 293.94 |

Particle Distribution Determined by Laser Diffraction with Dry Dispersal (2 bar Counterpressure):

Figures in μm (Details on the Measurement Method, See Under Methods)

| | Sample | | | | | | |
|---|---|---|---|---|---|---|---|
| | Dv10 | Dv20 | Dv25 | Dv30 | Dv50 | Dv75 | Dv90 |
| Vivapur ® 102 | 27.55 | 45.97 | 57.41 | 70.40 | 127.29 | 208.92 | 288.93 |

Particle Distribution Determined by Laser Diffraction with Dry Dispersal (3 bar Counterpressure):

Figures in μm (Details on the Measurement Method, See Under Methods)

| | Sample | | | | | | |
|---|---|---|---|---|---|---|---|
| | Dv10 | Dv20 | Dv25 | Dv30 | Dv50 | Dv75 | Dv90 |
| Vivapur ® 102 | 23.61 | 38.84 | 48.19 | 59.22 | 114.76 | 198.37 | 278.99 |

3. Other Materials
3.1 Propranolol HCl BP, EP, USP Batch No. M130302 (Changzhou Yabang Pharmaceutical Co., LTD., China)
3.2 Parteck® LUB MST (vegetable grade magnesium stearate) EMPROVE® exp Ph. Eur., BP, JP, NF, FCC Article No. 1.00663 (Merck KGaA, Germany)
3.3 Colloidal silicon dioxide, highly disperse, suitable for use as excipient; EMPROVE® exp Ph. Eur., NF, JP, E 551 Article No. 1.13126 (Merck KGaA, Germany)

Experimental Results

A) Aim of the Experiments:

Extended release oral active-ingredient formulations frequently have a complex structure. It is intended to show that the use of hydrophilic PVA grades as retarding polymer matrices enables the production of propranolol tablets having extended release of active ingredient (cumulative>80% release of active ingredient after 12 hours) by the simplest possible route. The experiments investigate what dependences the in-vitro release behaviour of these tablets has on the pH values of the release media, and how it is influenced by alcohol, possibly also accelerated. Suitable compositions for the intended use are those in which alcohol has no influence on the release behaviour and the release behaviour is independent of the pH.

These two properties are primary prerequisites for preventing any dose dumping effects from extended release formulations.

The applications PCT/EP2015/001355, (I14/067), PCT/EP2015/001356 (I 14/110) and PCT/EP2015/001357 (I 14/173) already filed have shown that only co-mixtures of ground polyvinyl alcohols (PVAs) of specific particle sizes with microcrystalline celluloses (MCCs) of specific particle sizes lead to good compressibility.

B) Summary of the Results:

With the following data, it can surprisingly be shown that propranolol tablets having extended release of active ingredient can be produced particularly simply using the co-mixtures described here as directly compressible retardation matrices, where it has surprisingly been found that
1. tablets having high hardnesses and low friabilities obtained even at low compression forces,
2. the release of active ingredient is independent of the pH of the release media used and
3. ethanol does not cause any change, in particular acceleration, in the release of active ingredient.

These are all advantages which simplify the development and production of extended release formulations of this type, but in particular also improve the medicament safety.

C) Procedure:
1. Preparation of the two co-mixtures PVA 26-88/MCC and PVA 40-88/MCC, mixing with the active ingredient and with further additives and compression at a compression force of 5, 10, 20 and 30 kN, and subsequent pharmaceutical formulation characterisation of the pressed products obtained.
2. Measurement of the in-vitro releases of propranolol from the extended release tablets in media having various pH values over a period of 12 hours.

The release data for the tablets from Examples A and B, which are obtained at a compression force of 10, 20 and 30 kN, are shown by way of example in the tables and graphs.
3. Measurement of the in-vitro release of propranolol from the extended release tablets in 0.1N HCl containing various amounts of ethanol.

The release data for the tablets from Examples A and B, which are obtained at a compression force of 10, 20 and 30 kN, are shown by way of example in the tables and graphs.

D) Experimental Results in Detail:
Re 1.: Preparation and pharmaceutical formulation characterisation of the propranolol extended release tablets:
a. Preparation of the co-mixtures of the two ground PVA grades 26-88 and 40-88 with microcrystalline cellulose (MCC) in the mixing ratio of 1:1. In the following examples, co-mixtures as have been described in the patent applications PCT/EP2015/001355, PCT/EP2015/001356 and PCT/EP2015/001357 are employed. These are co-mixtures of ground polyvinyl alcohols (PVAs) with microcrystalline celluloses (MCC) of specific particle sizes, where the particle sizes of the PVAs have been set by grinding.
b. 337.5 g of these co-mixtures are mixed with 160 g of propranolol HCl and 1.25 g of highly disperse silicon dioxide in a Turbula® mixer for 5 minutes. The mixture is then passed through an 800 μm hand sieve.
c. After addition of 1.25 g of Parteck® LUB MST, mixing is again carried out for 5 minutes. The mixture obtained as subsequently tabletted in a Korsch EK 0-DMS eccentric press to give tablets weighing 500 mg. The tablets produced in this way each comprise 160 mg of propranolol HCl per tablet.
d. The tablet characterisation is carried out with respect to the parameters tablet hardness, tablet weight, tablet thickness, tablet abrasion and ejection force required.

Composition (in % by Weight) Example A: with PVA 26-88 as Retardation Matrix

| | | Propranolol | Silicon | Magnesium |
|---|---|---|---|---|
| PVA 26-88* | MCC | HCl | dioxide | stearate |
| 33.75% | 33.75% | 32.0% | 0.25% | 0.25% |

*ground PVA

Composition (in % by Weight) Example B: with PVA 40-88 as Retardation Matrix

| | | Propranolol | Silicon | Magnesium |
|---|---|---|---|---|
| PVA 40-88* | MCC | HCl | dioxide | stearate |
| 33.75% | 33.75% | 32.0% | 0.25% | 0.25% |

*ground PVA

Tablet Characterisation

TABLE 1
Tableting data from Example A and Example B

| | A | | | | | | |
|---|---|---|---|---|---|---|---|
| Sample | Nominal | Actual | B | C | D | E | F |
| Example A | 5 | 5.8 | 64.6 | 522.4 | 5.5 | 0.47 | 219.6 |
| | 10 | 11.2 | 137.8 | 493.2 | 4.7 | 0.08 | 290.3 |
| | 20 | 19.9 | 232.1 | 495.6 | 4.5 | 0.05 | 296.6 |
| | 30 | 30.4 | 287.2 | 494.8 | 4.4 | 0.05 | 290.7 |
| Comparison B | 5 | 5.2 | 55.7 | 495.5 | 5.3 | 0.71 | 215.4 |
| | 10 | 9.5 | 120.0 | 502.4 | 4.9 | 0.02 | 303.2 |
| | 20 | 20.1 | 230.0 | 493.4 | 4.5 | 0.03 | 317.5 |
| | 30 | 30.7 | 289.3 | 504.7 | 4.5 | 0.02 | 314.1 |

Key:
A: Compression force [kN]
B: Tablet hardness after 1 day [N]
C: Tablet weight [mg]
D: Tablet thickness [mm]
E: Abrasion [%]
F: Ejection force (N)

FIG. 1 shows a graph of the compression force/tablet hardness profiles of the two examples for better illustration.

All tablets exhibit unusually high tablet hardnesses at all compression forces greater than/equal to 10 kN together with low abrasion after mechanical loading (low friability) and relatively low ejection forces.

There are virtually no differences in the tableting data between the tablets based on the matrices PVA 26-88 and PVA 40-88. In particular, the tablet hardnesses are virtually identical for the two PVA grades at the same compression forces.
Re 2.: In-vitro release from propranolol extended release tablets of Examples A and B in media having various pH values:
a) Measurement of the in-vitro release in phosphate buffer pH 6.8 over a period of 12 hours:
   testing of the pressed products obtained at a compression force of 10, 20 and 30 kN
b) Measurement of the in-vitro release in 0.1N HCl over a period of 12 hours:
   testing of the pressed products obtained at a compression force of 10, 20 and 30 kN
c) Measurement of the in-vitro release in HCl buffer pH 1.2 over a period of 12 hours:
   testing of the pressed products obtained at a compression force of 10, 20 and 30 kN
d) Measurement of the in-vitro release in 0.1N HCl over the period of 2 hours with subsequent re-buffering at pH 6.8 for 10 hours:
   testing of the pressed products obtained at a compression force of 10, 20 and 30 kN

TABLE 2a
In-vitro release data Example A (compression force 10 kN) in various media
The cumulative amounts of propranolol HCl (in %) released from the tablets
obtained at a compression force of 10 kN are shown.

| | | | | | | | | | | Re-buffering: | | |
|---|---|---|---|---|---|---|---|---|---|---|---|---|
| | | | | | | | | | | 2 hrs. 0.1N HCl | | |
| | Phosphate buffer | | | HCl | | | HCl buffer | | | then 10 hrs. | | |
| | pH 6.8 | | | 0.1N | | | pH 1.2 | | | pH 6.8 | | |
| Time | Min | Max | Mean | Min | Max | Mean | Min | Max | Mean | Min | Max | Mean |
| (hours) | (%) | (%) | (%) | (%) | (%) | (%) | (%) | (%) | (%) | (%) | (%) | (%) |
| 1 | 18 | 21 | 19 | 19 | 21 | 20 | 18 | 20 | 18 | 19 | 19 | 19 |
| 2 | 31 | 34 | 32 | 31 | 34 | 32 | 29 | 32 | 30 | 30 | 31 | 31 |
| 3 | 42 | 46 | 43 | 41 | 45 | 43 | 39 | 43 | 41 | 40 | 43 | 42 |
| 4 | 52 | 56 | 54 | 51 | 56 | 52 | 48 | 57 | 51 | 49 | 53 | 50 |
| 5 | 62 | 66 | 63 | 59 | 65 | 62 | 57 | 69 | 61 | 56 | 67 | 60 |
| 6 | 70 | 75 | 72 | 68 | 77 | 71 | 64 | 79 | 70 | 64 | 76 | 68 |
| 7 | 77 | 81 | 79 | 74 | 88 | 79 | 71 | 87 | 77 | 70 | 84 | 75 |
| 8 | 82 | 86 | 83 | 80 | 95 | 85 | 77 | 94 | 83 | 76 | 90 | 81 |
| 9 | 86 | 89 | 87 | 84 | 98 | 89 | 82 | 98 | 88 | 81 | 95 | 86 |
| 10 | 88 | 91 | 89 | 87 | 98 | 91 | 85 | 100 | 90 | 85 | 98 | 89 |
| 11 | 90 | 92 | 91 | 89 | 99 | 92 | 88 | 100 | 92 | 87 | 99 | 91 |
| 12 | 91 | 93 | 92 | 91 | 99 | 94 | 89 | 100 | 93 | 89 | 100 | 93 |

FIG. 2a shows a graph of the release data from Example A (tablets produced at a compression force of 10 kN) in the various media for better illustration.

TABLE 2b
In-vitro release data Example A (compression force 20 kN) in various media
The cumulative amounts of propranolol HCl (in %) released from the tablets
obtained at a compression force of 20 kN are shown.

| | | | | | | | | | | Re-buffering: | | |
|---|---|---|---|---|---|---|---|---|---|---|---|---|
| | | | | | | | | | | 2 hrs., 0.1N HCl, | | |
| | Phosphate buffer | | | HCl | | | HCl buffer | | | then 10 hrs. | | |
| | pH 6.8 | | | 0.1N | | | pH 1.2 | | | pH 6.8 | | |
| Time | Min | Max | Mean | Min | Max | Mean | Min | Max | Mean | Min | Max | Mean |
| (hours) | (%) | (%) | (%) | (%) | (%) | (%) | (%) | (%) | (%) | (%) | (%) | (%) |
| 1 | 17 | 18 | 18 | 18 | 19 | 18 | 17 | 18 | 17 | 17 | 21 | 19 |
| 2 | 29 | 31 | 30 | 30 | 32 | 30 | 28 | 29 | 29 | 29 | 35 | 31 |
| 3 | 40 | 42 | 41 | 40 | 43 | 41 | 38 | 40 | 39 | 39 | 46 | 42 |
| 4 | 50 | 52 | 51 | 49 | 52 | 50 | 47 | 49 | 48 | 47 | 55 | 50 |
| 5 | 59 | 62 | 60 | 57 | 61 | 59 | 55 | 58 | 56 | 55 | 63 | 58 |
| 6 | 66 | 70 | 68 | 65 | 69 | 66 | 62 | 65 | 63 | 62 | 71 | 65 |
| 7 | 73 | 77 | 75 | 71 | 75 | 73 | 69 | 72 | 70 | 68 | 77 | 71 |
| 8 | 79 | 83 | 81 | 77 | 81 | 78 | 75 | 78 | 76 | 73 | 82 | 77 |
| 9 | 84 | 88 | 85 | 81 | 85 | 83 | 79 | 83 | 81 | 78 | 87 | 82 |
| 10 | 86 | 90 | 88 | 84 | 89 | 86 | 83 | 87 | 85 | 82 | 91 | 85 |
| 11 | 88 | 92 | 90 | 87 | 91 | 88 | 86 | 90 | 87 | 85 | 94 | 88 |
| 12 | 90 | 93 | 91 | 89 | 93 | 90 | 88 | 92 | 90 | 88 | 96 | 91 |

FIG. 2b shows a graph of the release data from Example A (tablets produced at a compression force of 20 kN) in the various media for better illustration.

TABLE 2c
In-vitro release data Example A (compression force 30 kN) in various media
The cumulative amounts of propranolol HCl (in %) released from the tablets
obtained at a compression force of 30 kN are shown.

| | | | | | | | | | | Re-buffering: | | |
|---|---|---|---|---|---|---|---|---|---|---|---|---|
| | | | | | | | | | | 2 hrs. 0.1N HCl | | |
| | Phosphate buffer | | | HCl | | | HCl buffer | | | then 10 hrs. | | |
| | pH 6.8 | | | 0.1N | | | pH 1.2 | | | pH 6.8 | | |
| Time | Min | Max | Mean | Min | Max | Mean | Min | Max | Mean | Min | Max | Mean |
| (hours) | (%) | (%) | (%) | (%) | (%) | (%) | (%) | (%) | (%) | (%) | (%) | (%) |
| 1 | 17 | 18 | 18 | 17 | 19 | 18 | 17 | 18 | 17 | 18 | 18 | 18 |
| 2 | 29 | 30 | 30 | 29 | 31 | 30 | 28 | 30 | 28 | 29 | 31 | 30 |
| 3 | 39 | 42 | 40 | 39 | 42 | 41 | 37 | 40 | 38 | 39 | 41 | 40 |
| 4 | 49 | 51 | 50 | 48 | 51 | 50 | 46 | 49 | 47 | 48 | 52 | 49 |
| 5 | 57 | 60 | 58 | 56 | 60 | 58 | 54 | 58 | 56 | 56 | 62 | 58 |
| 6 | 64 | 68 | 66 | 64 | 67 | 65 | 61 | 66 | 63 | 63 | 70 | 65 |
| 7 | 70 | 75 | 72 | 70 | 74 | 72 | 67 | 73 | 69 | 69 | 77 | 71 |
| 8 | 75 | 80 | 78 | 75 | 79 | 77 | 73 | 78 | 75 | 74 | 83 | 76 |
| 9 | 80 | 84 | 82 | 80 | 84 | 82 | 78 | 83 | 80 | 78 | 87 | 81 |
| 10 | 83 | 87 | 85 | 84 | 88 | 86 | 82 | 87 | 84 | 82 | 90 | 84 |
| 11 | 85 | 89 | 87 | 86 | 90 | 88 | 85 | 91 | 87 | 84 | 92 | 87 |
| 12 | 87 | 90 | 89 | 88 | 93 | 91 | 87 | 93 | 89 | 86 | 94 | 89 |

FIG. 2c shows a graph of the release data from Example A (tablets produced at a compression force of 30 kN) in the various media for better illustration.

TABLE 2d
In-vitro release data Example B (compression force 10 kN) in various media
The cumulative amounts of propranolol HCl (in %) released from the tablets
obtained at a compression force of 10 kN are shown.

| | | | | | | | | | | Re-buffering: | | |
|---|---|---|---|---|---|---|---|---|---|---|---|---|
| | | | | | | | | | | 2 hrs. 0.1N HCl | | |
| | Phosphate buffer | | | HCl | | | HCl buffer | | | then 10 hrs. | | |
| | pH 6.8 | | | 0.1N | | | pH 1.2 | | | pH 6.8 | | |
| Time | Min | Max | Mean | Min | Max | Mean | Min | Max | Mean | Min | Max | Mean |
| (hours) | (%) | (%) | (%) | (%) | (%) | (%) | (%) | (%) | (%) | (%) | (%) | (%) |
| 1 | 18 | 18 | 18 | 19 | 19 | 19 | 19 | 20 | 20 | 18 | 20 | 19 |
| 2 | 30 | 30 | 30 | 30 | 31 | 31 | 30 | 33 | 32 | 30 | 31 | 30 |
| 3 | 40 | 41 | 41 | 40 | 42 | 41 | 40 | 47 | 44 | 40 | 41 | 41 |
| 4 | 50 | 51 | 51 | 49 | 56 | 52 | 51 | 57 | 54 | 48 | 53 | 51 |
| 5 | 59 | 61 | 59 | 58 | 67 | 63 | 62 | 67 | 64 | 55 | 62 | 59 |
| 6 | 66 | 70 | 67 | 66 | 76 | 72 | 69 | 75 | 71 | 61 | 70 | 67 |
| 7 | 73 | 77 | 74 | 73 | 84 | 80 | 75 | 82 | 78 | 68 | 78 | 74 |
| 8 | 78 | 83 | 80 | 80 | 91 | 87 | 80 | 89 | 85 | 74 | 84 | 80 |
| 9 | 82 | 87 | 84 | 86 | 96 | 92 | 85 | 94 | 90 | 78 | 90 | 86 |
| 10 | 85 | 91 | 88 | 90 | 98 | 95 | 88 | 97 | 94 | 82 | 94 | 90 |
| 11 | 88 | 93 | 90 | 92 | 98 | 96 | 91 | 99 | 96 | 86 | 97 | 93 |
| 12 | 90 | 94 | 92 | 94 | 99 | 97 | 93 | 100 | 97 | 88 | 99 | 95 |

FIG. 2d shows a graph of the release data from Example B (tablets produced at a compression force of 10 kN) in the various media for better illustration.

TABLE 2e
In-vitro release data Example B (compression force 20 kN) in various media
The cumulative amounts of propranolol HCl (in %) released from the tablets
obtained at a compression force of 20 kN are shown.

| | | | | | | | | | | Re-buffering: | | |
|---|---|---|---|---|---|---|---|---|---|---|---|---|
| | | | | | | | | | | 2 hrs. 0.1N HCl | | |
| | Phosphate buffer | | | HCl | | | HCl buffer | | | then 10 hrs. | | |
| | pH 6.8 | | | 0.1N | | | pH 1.2 | | | pH 6.8 | | |
| Time | Min | Max | Mean | Min | Max | Mean | Min | Max | Mean | Min | Max | Mean |
| (hours) | (%) | (%) | (%) | (%) | (%) | (%) | (%) | (%) | (%) | (%) | (%) | (%) |
| 1 | 15 | 16 | 16 | 17 | 18 | 18 | 16 | 17 | 17 | 16 | 17 | 17 |
| 2 | 25 | 27 | 26 | 28 | 30 | 29 | 27 | 28 | 27 | 27 | 28 | 28 |
| 3 | 34 | 37 | 35 | 38 | 40 | 39 | 36 | 38 | 37 | 36 | 38 | 37 |
| 4 | 42 | 46 | 43 | 47 | 49 | 48 | 44 | 47 | 45 | 43 | 46 | 45 |
| 5 | 49 | 53 | 51 | 55 | 57 | 56 | 51 | 56 | 53 | 49 | 53 | 51 |
| 6 | 55 | 60 | 57 | 62 | 65 | 63 | 58 | 63 | 60 | 55 | 60 | 58 |
| 7 | 61 | 66 | 63 | 68 | 72 | 70 | 64 | 70 | 66 | 61 | 66 | 63 |
| 8 | 66 | 72 | 68 | 74 | 78 | 76 | 70 | 76 | 72 | 66 | 71 | 69 |
| 9 | 71 | 77 | 73 | 79 | 83 | 81 | 75 | 81 | 77 | 70 | 76 | 73 |
| 10 | 75 | 81 | 78 | 83 | 87 | 85 | 79 | 85 | 81 | 74 | 81 | 77 |
| 11 | 78 | 85 | 81 | 86 | 91 | 89 | 83 | 88 | 85 | 78 | 84 | 81 |
| 12 | 81 | 88 | 84 | 89 | 94 | 91 | 85 | 91 | 88 | 81 | 88 | 84 |

FIG. 2e shows a graph of the release data from Example B (tablets produced at a compression force of 20 kN) in the various media for better illustration.

TABLE 2f
In-vitro release data Example B (compression force 30 kN) in various media
The cumulative amounts of propranolol HCl (in %) released from the tablets
obtained at a compression force of 30 kN are shown.

| | | | | | | | | | | Re-buffering: | | |
|---|---|---|---|---|---|---|---|---|---|---|---|---|
| | | | | | | | | | | 2 hrs. 0.1N HCl | | |
| | Phosphate buffer | | | HCl | | | HCl buffer | | | then 10 hrs. | | |
| | pH 6.8 | | | 0.1N | | | pH 1.2 | | | pH 6.8 | | |
| Time | Min | Max | Mean | Min | Max | Mean | Min | Max | Mean | Min | Max | Mean |
| (hours) | (%) | (%) | (%) | (%) | (%) | (%) | (%) | (%) | (%) | (%) | (%) | (%) |
| 1 | 16 | 16 | 16 | 18 | 18 | 18 | 16 | 17 | 17 | 16 | 17 | 17 |
| 2 | 26 | 27 | 27 | 29 | 30 | 29 | 27 | 28 | 27 | 27 | 28 | 27 |
| 3 | 36 | 36 | 36 | 39 | 40 | 39 | 36 | 38 | 37 | 35 | 38 | 36 |
| 4 | 44 | 45 | 44 | 47 | 49 | 48 | 44 | 47 | 45 | 42 | 46 | 44 |
| 5 | 51 | 52 | 51 | 55 | 58 | 57 | 52 | 55 | 53 | 49 | 54 | 51 |
| 6 | 57 | 59 | 58 | 63 | 66 | 64 | 58 | 62 | 60 | 54 | 60 | 57 |
| 7 | 63 | 65 | 64 | 69 | 73 | 71 | 64 | 69 | 66 | 60 | 66 | 62 |
| 8 | 68 | 70 | 69 | 75 | 78 | 77 | 70 | 75 | 72 | 65 | 72 | 68 |
| 9 | 73 | 75 | 74 | 80 | 84 | 82 | 75 | 80 | 77 | 69 | 76 | 72 |
| 10 | 77 | 79 | 78 | 85 | 88 | 86 | 79 | 85 | 81 | 73 | 81 | 76 |
| 11 | 80 | 83 | 82 | 88 | 92 | 90 | 83 | 89 | 85 | 76 | 84 | 80 |
| 12 | 83 | 86 | 85 | 91 | 95 | 93 | 86 | 92 | 88 | 80 | 87 | 83 |

FIG. 2f shows a graph of the release data from Example B (tablets produced at a compression force of 30 kN) in the various media for better illustration.

The data from Tables 2a to 2f show that the in-vitro release of propranolol in both Examples A and B is virtually independent of the release medium, in particular is independent of the pH of the release medium.
Re 3.: In-vitro release from propranolol extended release tablets of Examples A and B in media containing various amounts of alcohol.
a) Measurement of the in-vitro release in 0.1N HCl over 12 hours:
   Testing of the pressed products obtained at a compression force of 10, 20 and 30 kN
b) Measurement of the in-vitro release in 0.1N HCl with 40% by vol. of ethanol over 12 hours:
   Testing of the pressed products obtained at a compression force of 10, 20 and 30 kN
c) Measurement of the in-vitro release in 0.1N HCl with 20% by vol. of ethanol over 12 hours:
   Testing of the pressed products obtained at a compression force of 10, 20 and 30 kN
d) Measurement of the in-vitro release in 0.1N HCl with 5% by vol. of ethanol over 12 hours:
   Testing of the pressed products obtained at a compression force of 10, 20 and 30 kN

TABLE 3a
In-vitro release data Example A (compression force 10 kN) in
0.1N HCl with addition of various amounts of ethanol
The cumulative amounts of propranolol HCl (in %) released from the tablets
obtained at a compression force of 10 kN are shown.

| | HCl | | | Ethanol | | | Ethanol | | | Ethanol | | |
|---|---|---|---|---|---|---|---|---|---|---|---|---|
| | 0.1N | | | 40% by vol. | | | 20% by vol. | | | 5% by vol. | | |
| Time | Min | Max | Mean | Min | Max | Mean | Min | Max | Mean | Min | Max | Mean |
| (hours) | (%) | (%) | (%) | (%) | (%) | (%) | (%) | (%) | (%) | (%) | (%) | (%) |
| 1 | 19 | 21 | 20 | 19 | 19 | 19 | 19 | 21 | 19 | 19 | 20 | 20 |
| 2 | 31 | 34 | 32 | 32 | 32 | 32 | 31 | 35 | 33 | 32 | 32 | 32 |
| 3 | 41 | 45 | 43 | 44 | 44 | 44 | 42 | 47 | 44 | 43 | 43 | 43 |
| 4 | 51 | 56 | 52 | 54 | 55 | 55 | 52 | 61 | 56 | 54 | 54 | 54 |
| 5 | 59 | 65 | 62 | 64 | 66 | 65 | 62 | 73 | 66 | 63 | 63 | 63 |
| 6 | 68 | 77 | 71 | 75 | 79 | 77 | 72 | 83 | 76 | 72 | 72 | 72 |
| 7 | 74 | 88 | 79 | 88 | 90 | 89 | 80 | 92 | 85 | 79 | 79 | 79 |
| 8 | 80 | 95 | 85 | 95 | 98 | 96 | 86 | 98 | 92 | 84 | 85 | 84 |
| 9 | 84 | 98 | 89 | 99 | 101 | 100 | 90 | 100 | 96 | 88 | 89 | 89 |
| 10 | 87 | 98 | 91 | 101 | 103 | 102 | 95 | 101 | 99 | 90 | 93 | 91 |
| 11 | 89 | 99 | 92 | 102 | 104 | 103 | 98 | 101 | 100 | 92 | 95 | 94 |
| 12 | 91 | 99 | 94 | 102 | 104 | 103 | 100 | 102 | 101 | 95 | 98 | 97 |

FIG. 3a shows a graph of the release data from Example A (tablets produced at a compression force of 10 kN) in 0.1N HCl compared with the ethanol-containing media.

TABLE 3b
In-vitro release data Example A (compression force 20 kN) in
0.1N HCl with addition of various amounts of ethanol
The cumulative amounts of propranolol HCl (in %) released from the tablets
obtained at a compression force of 20 kN are shown.

| | HCl | | | Ethanol | | | Ethanol | | | Ethanol | | |
|---|---|---|---|---|---|---|---|---|---|---|---|---|
| | 0.1N | | | 40% by vol. | | | 20% by vol. | | | 5% by vol. | | |
| Time | Min | Max | Mean | Min | Max | Mean | Min | Max | Mean | Min | Max | Mean |
| (hours) | (%) | (%) | (%) | (%) | (%) | (%) | (%) | (%) | (%) | (%) | (%) | (%) |
| 1 | 18 | 19 | 18 | 18 | 19 | 19 | 18 | 18 | 18 | 18 | 22 | 20 |
| 2 | 30 | 32 | 30 | 31 | 32 | 31 | 29 | 31 | 30 | 31 | 39 | 33 |
| 3 | 40 | 43 | 41 | 42 | 44 | 43 | 40 | 43 | 41 | 41 | 53 | 45 |
| 4 | 49 | 52 | 50 | 52 | 55 | 53 | 50 | 53 | 51 | 51 | 67 | 56 |
| 5 | 57 | 61 | 59 | 61 | 64 | 62 | 59 | 62 | 60 | 60 | 74 | 65 |
| 6 | 65 | 69 | 66 | 69 | 73 | 71 | 67 | 70 | 68 | 68 | 80 | 72 |
| 7 | 71 | 75 | 73 | 76 | 81 | 78 | 74 | 77 | 75 | 75 | 85 | 78 |
| 8 | 77 | 81 | 78 | 83 | 88 | 85 | 79 | 83 | 81 | 80 | 90 | 84 |
| 9 | 81 | 85 | 83 | 88 | 93 | 90 | 84 | 88 | 86 | 85 | 93 | 88 |
| 10 | 84 | 89 | 86 | 91 | 96 | 93 | 87 | 91 | 89 | 88 | 95 | 91 |
| 11 | 87 | 91 | 88 | 94 | 98 | 95 | 90 | 93 | 91 | 90 | 97 | 93 |
| 12 | 89 | 93 | 90 | 95 | 99 | 97 | 93 | 95 | 94 | 92 | 99 | 95 |

FIG. 3b shows a graph of the release data from Example A (tablets produced at a compression force of 20 kN) in 0.1N HCl compared with the ethanol-containing media.

TABLE 3c
In-vitro release data Example A (compression force 30 kN) in
0.1N HCl with addition of various amounts of ethanol
The cumulative amounts of propranolol HCl (in %) released from the tablets
obtained at a compression force of 30 kN are shown.

| | HCl | | | Ethanol | | | Ethanol | | | Ethanol | | |
|---|---|---|---|---|---|---|---|---|---|---|---|---|
| | 0.1N | | | 40% by vol. | | | 20% by vol. | | | 5% by vol. | | |
| Time | Min | Max | Mean | Min | Max | Mean | Min | Max | Mean | Min | Max | Mean |
| (hours) | (%) | (%) | (%) | (%) | (%) | (%) | (%) | (%) | (%) | (%) | (%) | (%) |
| 1 | 17 | 19 | 18 | 18 | 19 | 19 | 18 | 19 | 18 | 18 | 20 | 19 |
| 2 | 29 | 31 | 30 | 30 | 32 | 31 | 30 | 32 | 31 | 30 | 33 | 32 |
| 3 | 39 | 42 | 41 | 41 | 44 | 43 | 41 | 43 | 42 | 40 | 45 | 43 |
| 4 | 48 | 51 | 50 | 51 | 54 | 53 | 51 | 53 | 52 | 49 | 55 | 53 |
| 5 | 56 | 60 | 58 | 59 | 64 | 62 | 60 | 62 | 61 | 57 | 65 | 62 |
| 6 | 64 | 67 | 65 | 67 | 72 | 70 | 67 | 71 | 69 | 64 | 73 | 69 |
| 7 | 70 | 74 | 72 | 74 | 80 | 78 | 74 | 78 | 76 | 70 | 81 | 76 |
| 8 | 75 | 79 | 77 | 80 | 86 | 84 | 80 | 84 | 82 | 75 | 87 | 82 |
| 9 | 80 | 84 | 82 | 85 | 91 | 89 | 85 | 89 | 87 | 80 | 92 | 87 |
| 10 | 84 | 88 | 86 | 89 | 95 | 92 | 89 | 93 | 91 | 84 | 95 | 90 |
| 11 | 86 | 90 | 88 | 91 | 97 | 95 | 91 | 95 | 93 | 86 | 97 | 92 |
| 12 | 88 | 93 | 91 | 94 | 99 | 97 | 93 | 97 | 95 | 89 | 99 | 94 |

FIG. 3c shows a graph of the release data from Example A (tablets produced at a compression force of 30 kN) in 0.1N HCl compared with the ethanol-containing media.

TABLE 3d
In-vitro release data Example B (compression force 10 kN) in
0.1N HCl with addition of various amounts of ethanol
The cumulative amounts of propranolol HCl (in %) released from the tablets
obtained at a compression force of 10 kN are shown.

| | HCl | | | Ethanol | | | Ethanol | | | Ethanol | | |
|---|---|---|---|---|---|---|---|---|---|---|---|---|
| | 0.1N | | | 40% by vol. | | | 20% by vol. | | | 5% by vol. | | |
| Time | Min | Max | Mean | Min | Max | Mean | Min | Max | Mean | Min | Max | Mean |
| (hours) | (%) | (%) | (%) | (%) | (%) | (%) | (%) | (%) | (%) | (%) | (%) | (%) |
| 1 | 19 | 19 | 19 | 19 | 21 | 20 | 18 | 20 | 19 | 18 | 19 | 19 |
| 2 | 30 | 31 | 31 | 31 | 35 | 33 | 30 | 34 | 32 | 29 | 31 | 31 |
| 3 | 40 | 42 | 41 | 42 | 52 | 45 | 40 | 47 | 43 | 40 | 42 | 41 |
| 4 | 49 | 56 | 52 | 52 | 65 | 57 | 49 | 59 | 53 | 49 | 52 | 51 |
| 5 | 58 | 67 | 63 | 62 | 75 | 66 | 58 | 69 | 62 | 58 | 65 | 61 |
| 6 | 66 | 76 | 72 | 74 | 84 | 78 | 65 | 78 | 72 | 66 | 77 | 72 |
| 7 | 73 | 84 | 80 | 85 | 91 | 87 | 72 | 85 | 79 | 73 | 85 | 81 |
| 8 | 80 | 91 | 87 | 92 | 99 | 94 | 77 | 91 | 86 | 78 | 92 | 88 |
| 9 | 86 | 96 | 92 | 96 | 101 | 98 | 82 | 96 | 91 | 83 | 98 | 93 |
| 10 | 90 | 98 | 95 | 100 | 103 | 101 | 85 | 99 | 94 | 87 | 101 | 96 |
| 11 | 92 | 98 | 96 | 101 | 104 | 103 | 88 | 100 | 96 | 90 | 101 | 97 |
| 12 | 94 | 99 | 97 | 102 | 104 | 103 | 90 | 101 | 97 | 92 | 102 | 98 |

FIG. 3d shows a graph of the release data from Example B (tablets produced at a compression force of 10 kN) in 0.1N HCl compared with the ethanol-containing media.

TABLE 3e
In-vitro release data Example B (compression force 20 kN) in
0.1N HCl with addition of various amounts of ethanol
The cumulative amounts of propranolol HCl (in %) released from the tablets
obtained at a compression force of 20 kN are shown.

| | HCl | | | Ethanol | | | Ethanol | | | Ethanol | | |
|---|---|---|---|---|---|---|---|---|---|---|---|---|
| | 0.1N | | | 40 Vol-% | | | 20 Vol-% | | | 5 Vol-% | | |
| Time | Min | Max | Mean | Min | Max | Mean | Min | Max | Mean | Min | Max | Mean |
| (hours) | (%) | (%) | (%) | (%) | (%) | (%) | (%) | (%) | (%) | (%) | (%) | (%) |
| 1 | 17 | 18 | 18 | 18 | 24 | 20 | 17 | 21 | 19 | 17 | 19 | 18 |
| 2 | 28 | 30 | 29 | 29 | 41 | 33 | 27 | 37 | 32 | 29 | 31 | 29 |
| 3 | 38 | 40 | 39 | 40 | 55 | 45 | 37 | 51 | 45 | 38 | 41 | 39 |
| 4 | 47 | 49 | 48 | 49 | 64 | 55 | 45 | 65 | 56 | 47 | 51 | 49 |
| 5 | 55 | 57 | 56 | 58 | 72 | 63 | 53 | 79 | 67 | 56 | 59 | 57 |
| 6 | 62 | 65 | 63 | 66 | 80 | 71 | 60 | 87 | 75 | 63 | 66 | 64 |
| 7 | 68 | 72 | 70 | 73 | 87 | 79 | 66 | 93 | 82 | 69 | 73 | 71 |
| 8 | 74 | 78 | 76 | 80 | 91 | 85 | 72 | 97 | 88 | 75 | 79 | 76 |
| 9 | 79 | 83 | 81 | 86 | 95 | 90 | 77 | 99 | 92 | 80 | 84 | 81 |
| 10 | 83 | 87 | 85 | 90 | 97 | 94 | 81 | 100 | 94 | 84 | 89 | 86 |
| 11 | 86 | 91 | 89 | 93 | 98 | 96 | 85 | 100 | 95 | 87 | 92 | 89 |
| 12 | 89 | 94 | 91 | 95 | 99 | 98 | 88 | 101 | 96 | 89 | 95 | 92 |

FIG. 3e shows a graph of the release data from Example B (tablets produced at a compression force of 20 kN) in 0.1N HCl compared with the ethanol-containing media.

TABLE 3f
In-vitro release data Example B (compression force 30 kN) in 0.1N
HCl with addition of various amounts of ethanol_{—}
The cumulative amounts of propranolol HCl (in %) released from the tablets
obtained at a compression force of 30 kN are shown.

| | HCl | | | Ethanol | | | Ethanol | | | Ethanol | | |
|---|---|---|---|---|---|---|---|---|---|---|---|---|
| | 0.1N | | | 40% by vol. | | | 20% by vol. | | | 5% by vol. | | |
| Time | Min | Max | Mean | Min | Max | Mean | Min | Max | Mean | Min | Max | Mean |
| (hours) | (%) | (%) | (%) | (%) | (%) | (%) | (%) | (%) | (%) | (%) | (%) | (%) |
| 1 | 18 | 18 | 18 | 18 | 20 | 19 | 17 | 18 | 17 | 17 | 19 | 18 |
| 2 | 29 | 30 | 29 | 29 | 34 | 31 | 27 | 30 | 29 | 28 | 32 | 30 |
| 3 | 39 | 40 | 39 | 39 | 47 | 43 | 36 | 41 | 39 | 38 | 44 | 41 |
| 4 | 47 | 49 | 48 | 49 | 60 | 53 | 45 | 51 | 48 | 46 | 55 | 51 |
| 5 | 55 | 58 | 57 | 57 | 71 | 63 | 53 | 61 | 57 | 54 | 65 | 60 |
| 6 | 63 | 66 | 64 | 65 | 78 | 70 | 60 | 70 | 65 | 61 | 73 | 67 |
| 7 | 69 | 73 | 71 | 72 | 84 | 77 | 66 | 77 | 72 | 67 | 80 | 74 |
| 8 | 75 | 78 | 77 | 79 | 90 | 83 | 72 | 83 | 78 | 72 | 86 | 79 |
| 9 | 80 | 84 | 82 | 84 | 94 | 88 | 77 | 90 | 83 | 77 | 90 | 85 |
| 10 | 85 | 88 | 86 | 89 | 97 | 93 | 82 | 94 | 88 | 82 | 94 | 89 |
| 11 | 88 | 92 | 90 | 92 | 98 | 95 | 86 | 97 | 92 | 87 | 96 | 93 |
| 12 | 91 | 95 | 93 | 94 | 99 | 97 | 90 | 99 | 94 | 91 | 99 | 96 |

FIG. 3f shows a graph of the release data from Example B (tablets produced at a compression force of 30 kN) in 0.1N HCl compared with the ethanol-containing media.

The data from Tables 3a to 3f show that the in-vitro release of propranolol in the two Examples A and B is also not significantly changed by addition of alcohol in the range from 5 to 40% by vol., even over 12 hours. In particular, no significantly accelerated release of active ingredient takes place, as would perhaps have been expected.

### LIST OF FIGURES

FIG. 1: Compression force/tablet hardness profiles of Examples A and B (from Table 1)

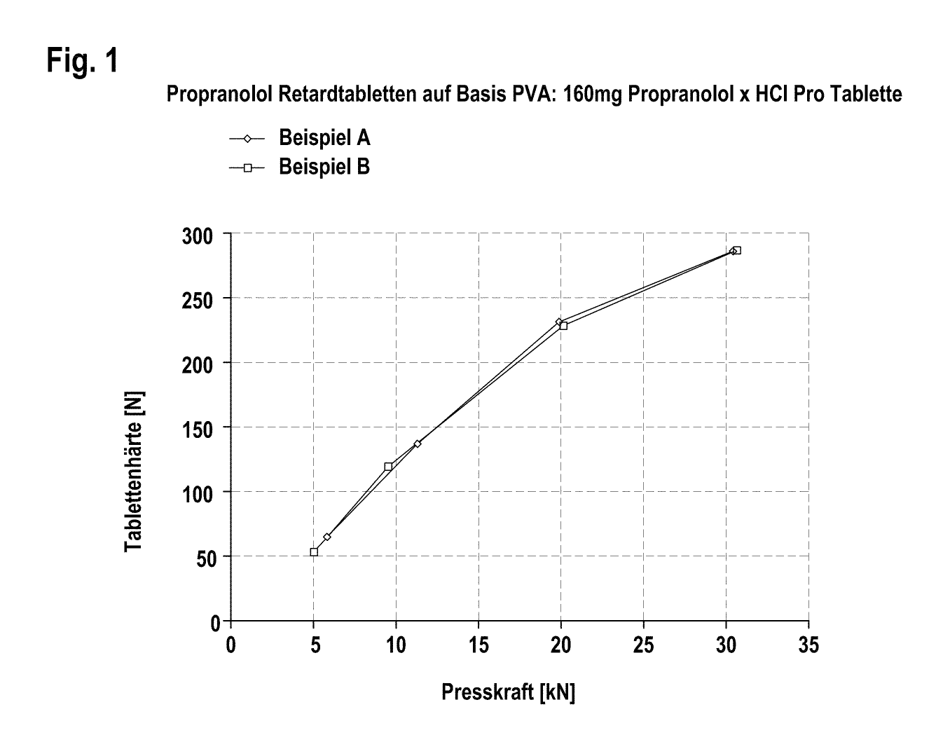

FIG. 2a: Release data Example A (compression force 10 kN) in media having different pH values (from Table 2a)

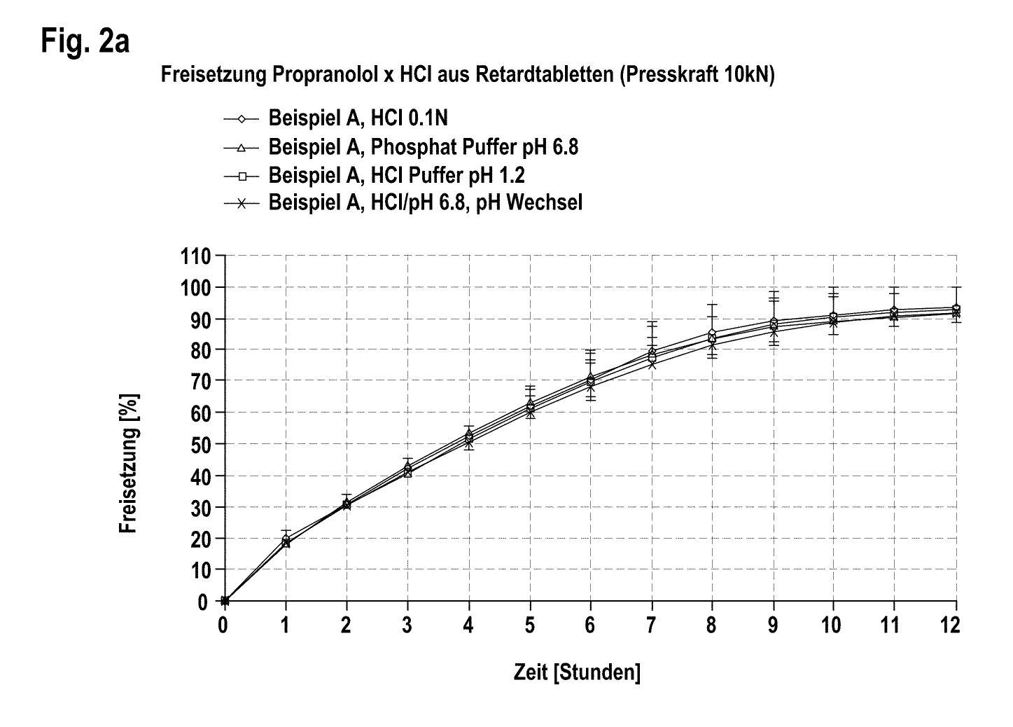

FIG. 2b: Release data Example A (compression force 20 kN) in media having different pH values (from Table 2b)

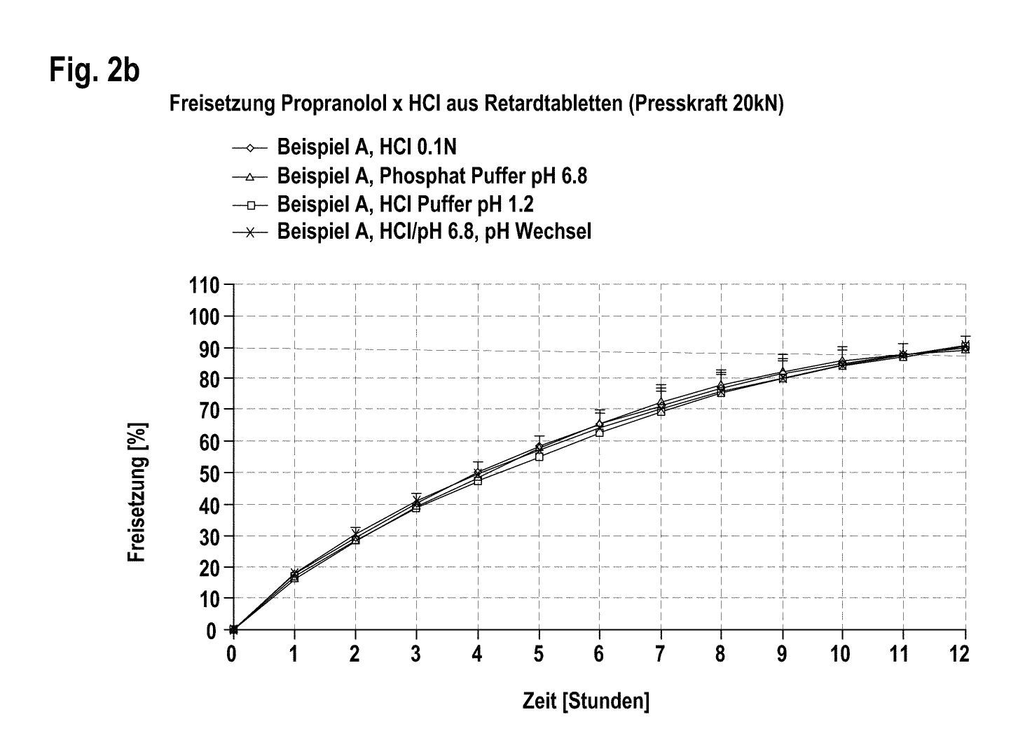

FIG. 2c: Release data Example A (compression force 30 kN) in media having different pH values (from Table 2c)

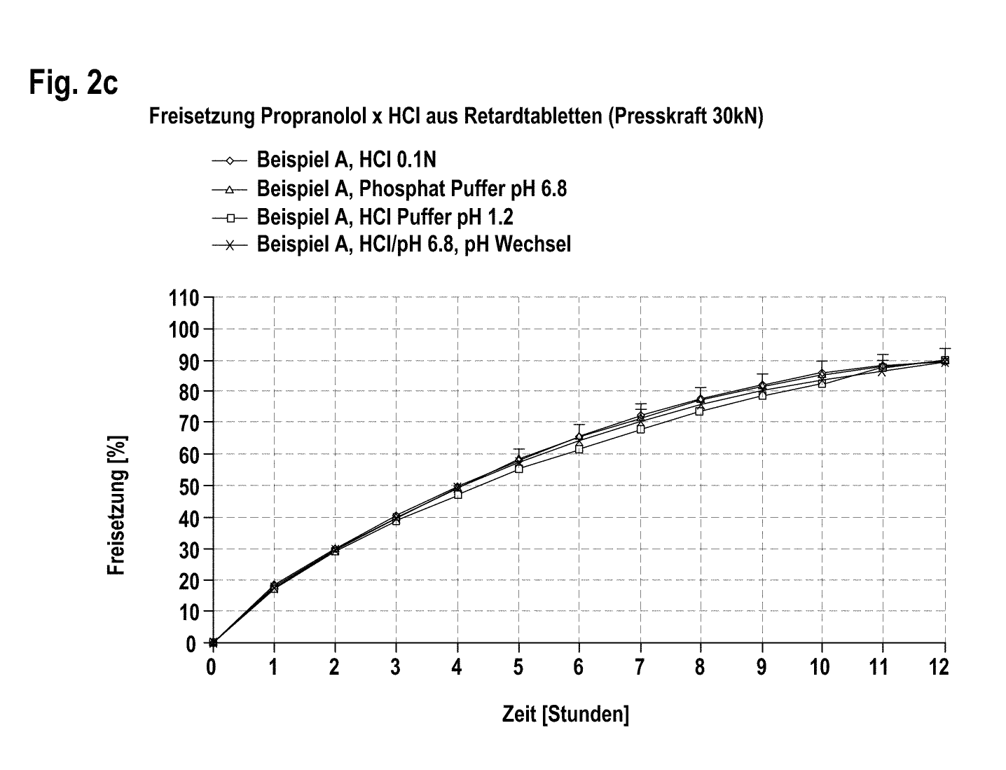

FIG. 2d: Release data Example B (compression force 10 kN) in media having different pH values (from Table 2d)

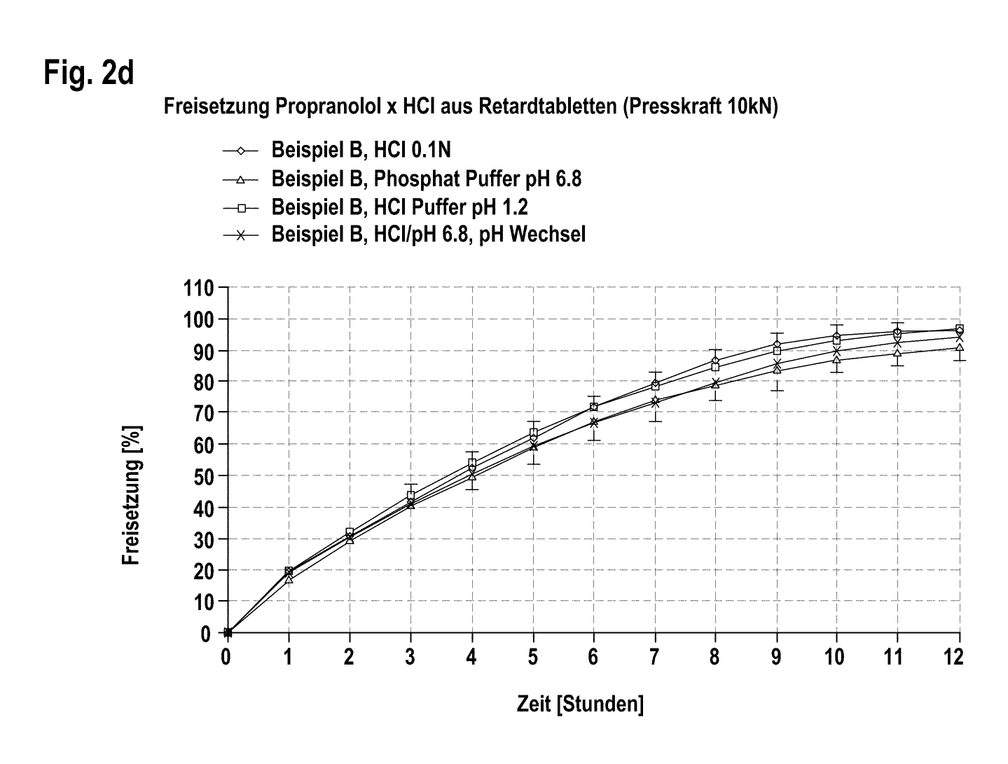

FIG. 2e: Release data Example B (compression force 20 kN) in media having different pH values (from Table 2e)

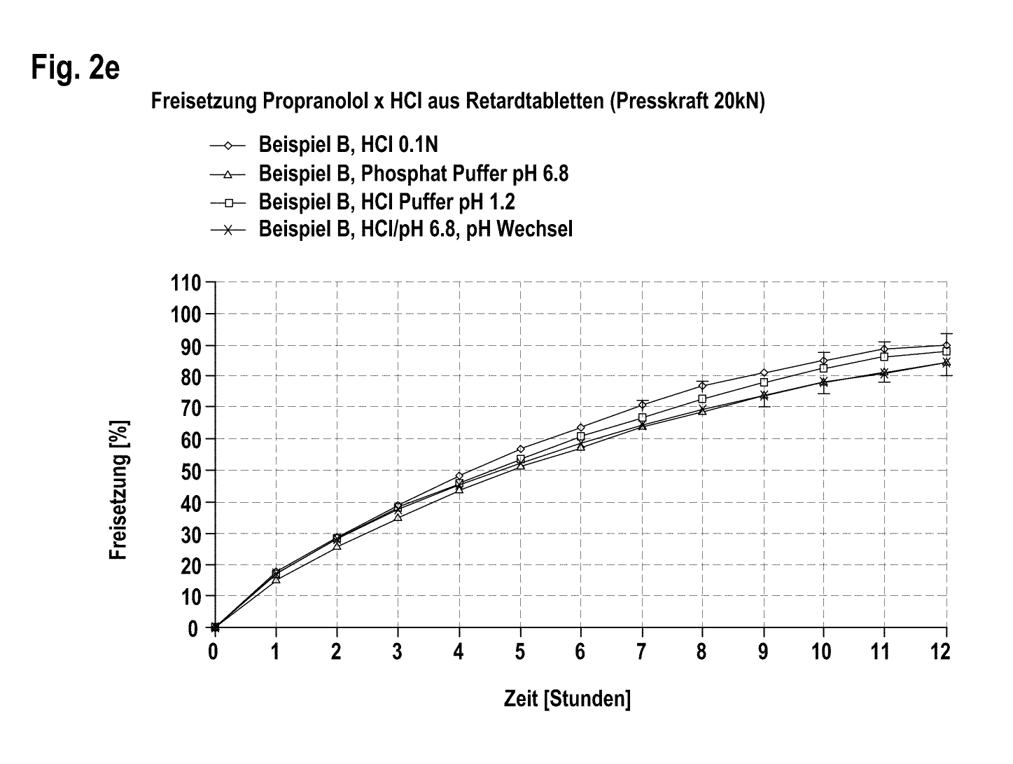

FIG. 2f: Release data Example B (compression force 30 kN) in media having different pH values (from Table 2f)

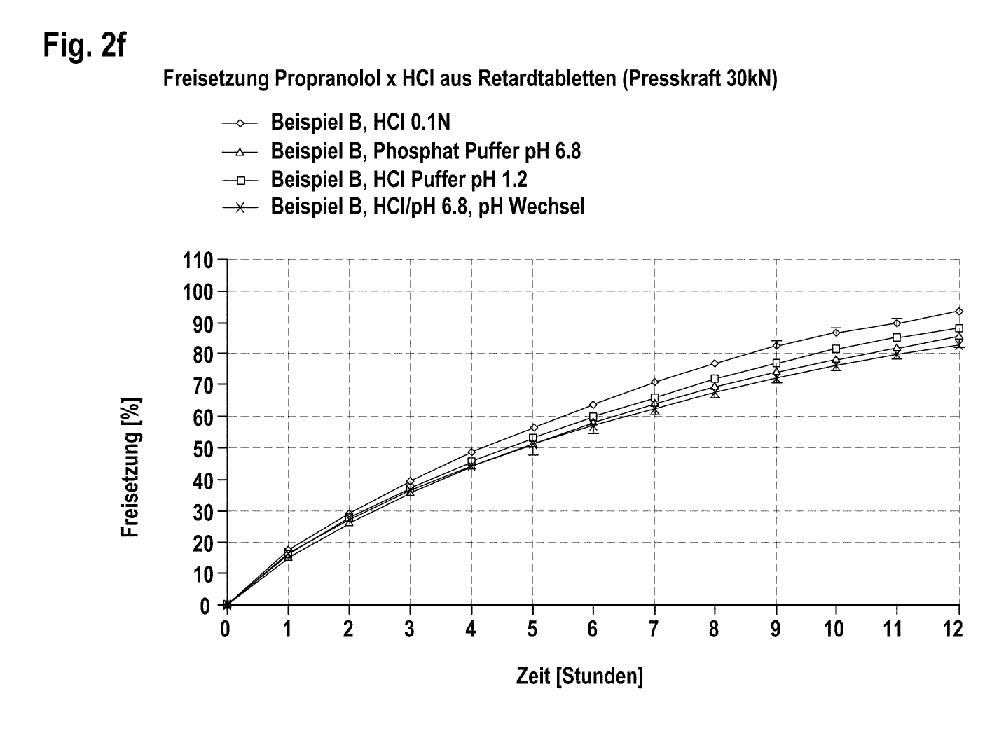

FIG. 3a: Release data Example A (compression force 10 kN) in 0.1N HCl with addition of various amounts of ethanol (from Table 3a)

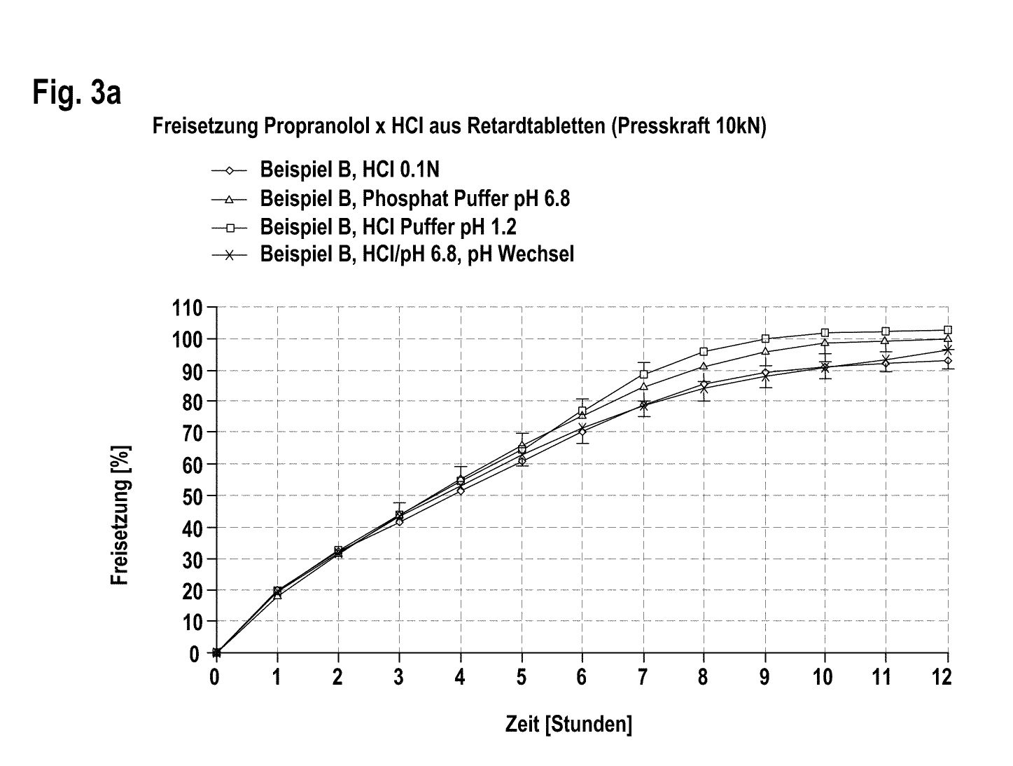

FIG. 3b: Release data Example A (compression force 20 kN) in 0.1N HCl with addition of various amounts of ethanol (from Table 3b)

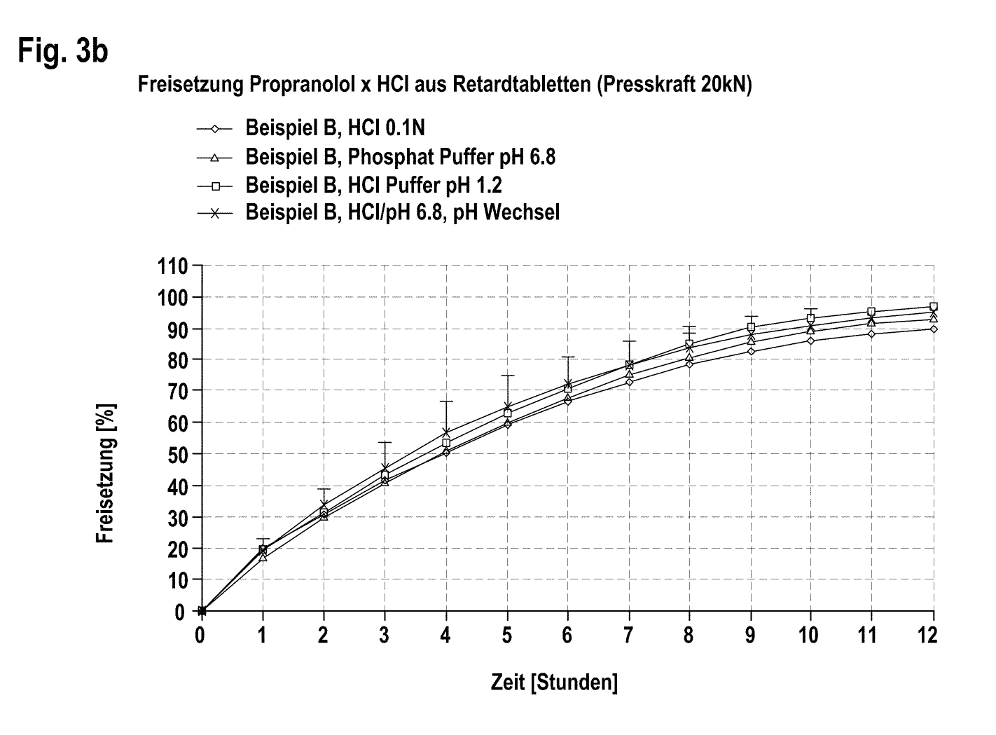

FIG. 3c: Release data Example A (compression force 30 kN) in 0.1N HCl with addition of various amounts of ethanol (from Table 3c)

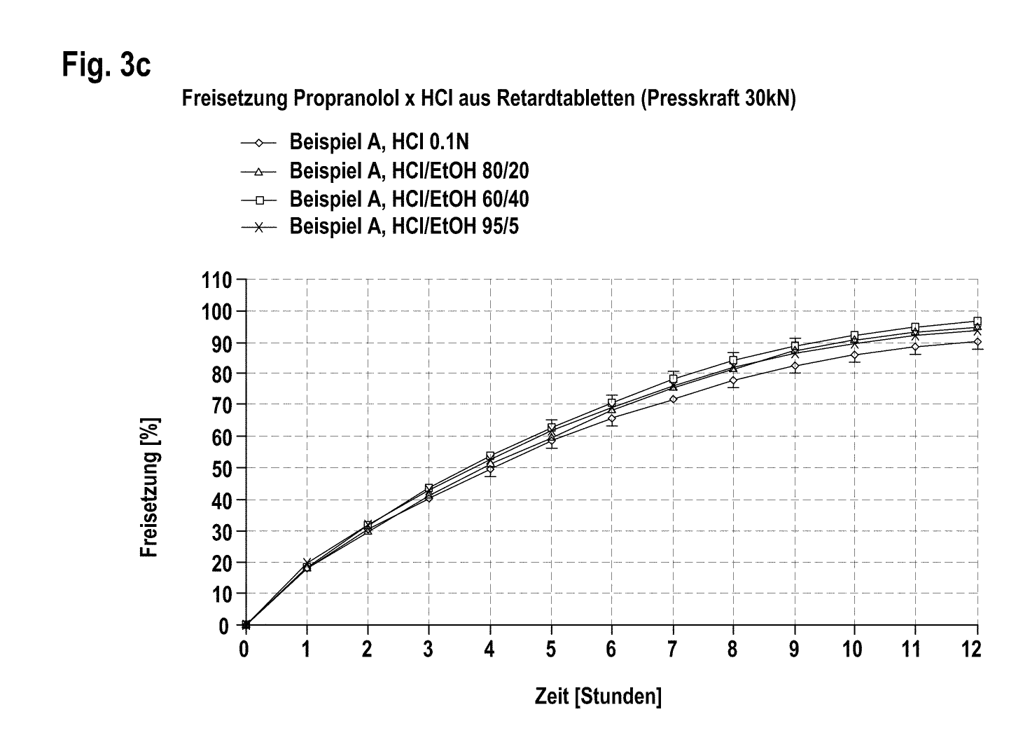

FIG. 3d: Release data Example B (compression force 10 kN) in 0.1N HCl with addition of various amounts of ethanol (from Table 3d)

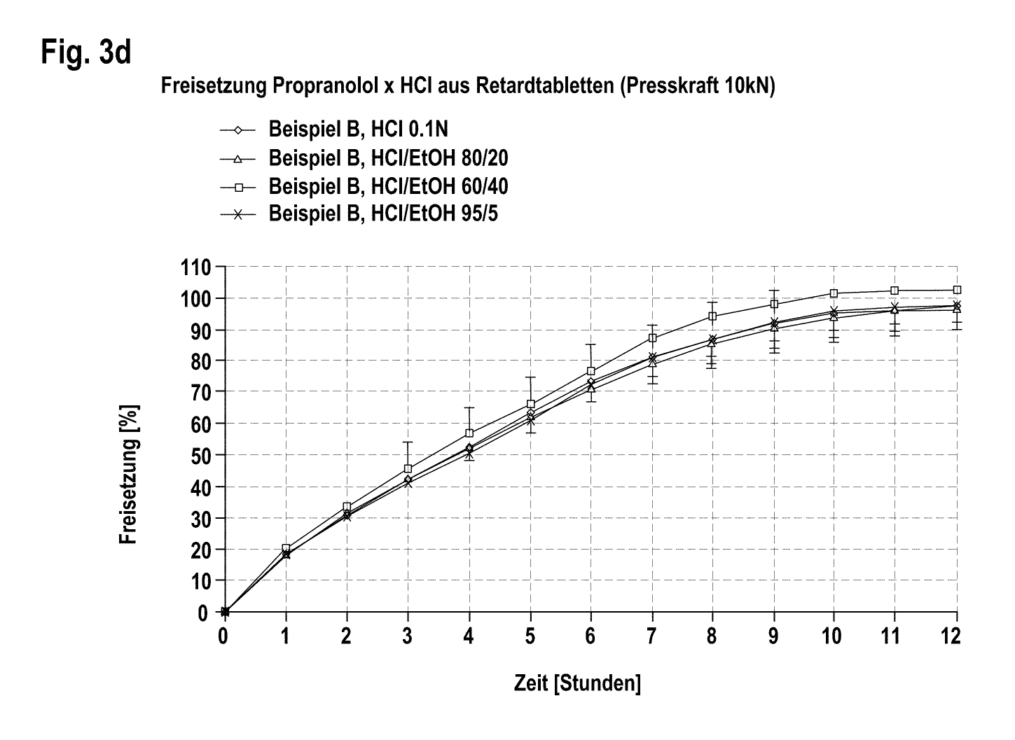

FIG. 3e: Release data Example B (compression force 20 kN) in 0.1N HCl with addition of various amounts of ethanol (from Table 3e)

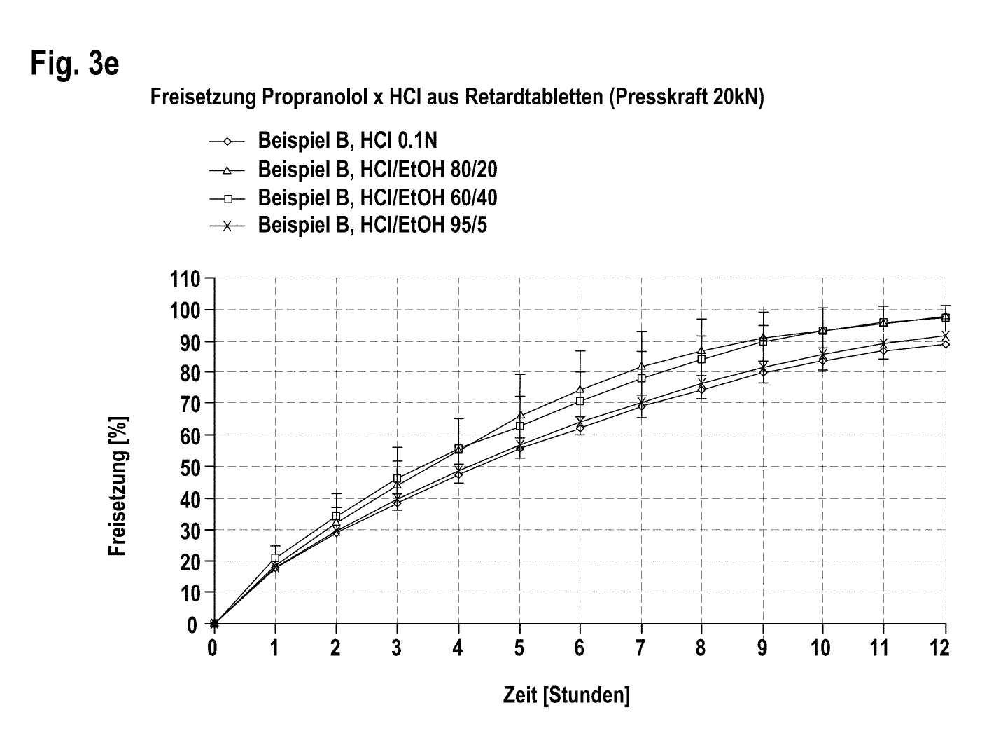

FIG. 3f: Release data Example B (compression force 30 kN) in 0.1N HCl with addition of various amounts of ethanol (from Table 3f)

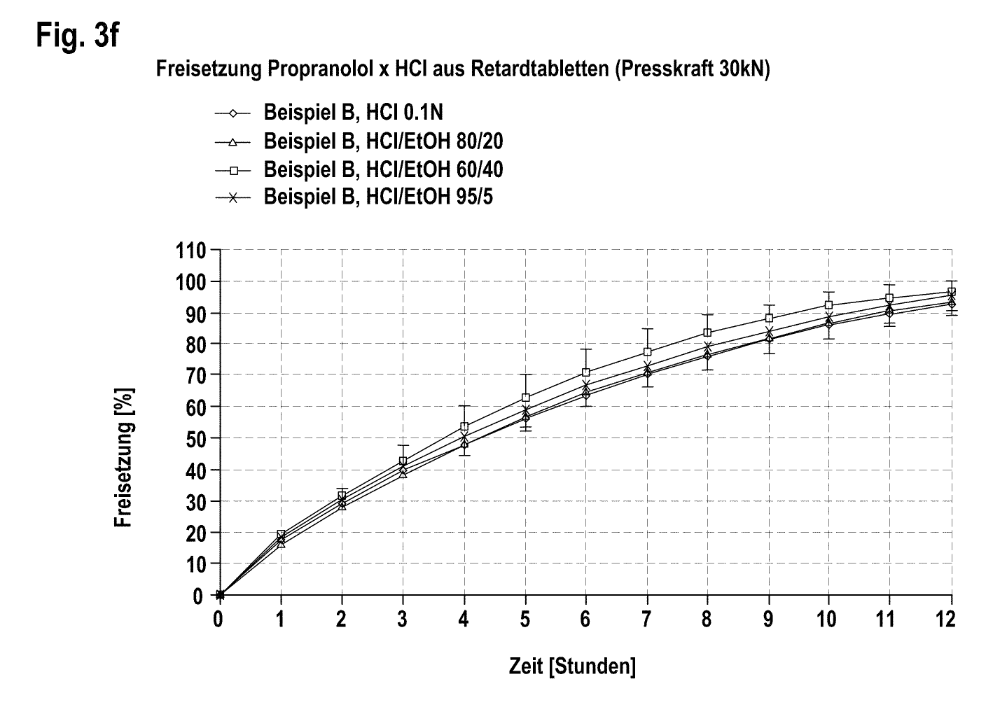

1. Formulations having extended release of active ingredient, comprising a pharmaceutical active ingredient and polyvinyl alcohols (PVAs) as matrix, where the release of the active ingredient takes place over a therapeutically relevant time period independently of the composition of the release medium.

2. Formulations according to claim 1 which have a release of active ingredient which is independent of the pH and ethanol content of the release medium.

3. Formulations according to claim 1 which have an independent active-ingredient release behaviour at a pH in the range from 1 to 7 in the release medium.

4. Formulations according to claim 1 which have an independent active-ingredient release behaviour at an alcohol content in the range from 5 to 40% by vol. in the release medium.

5. Formulations according to claim 1, comprising a pharmaceutical active ingredient and PVAs having an average particle size <100 μm.

6. Formulations according to claim 1, comprising a pharmaceutical active ingredient and a combination (co-mixture) of PVAs with a microcrystalline cellulose.

7. Formulations according to claim 1, comprising microcrystalline cellulose having an average particle size <150 μm, preferably having an average particle size in the range from 100 to 140 μm.

8. Formulations according to claim 1, characterised in that they comprise co-mixtures of PVA and microcrystalline celluloses in the ratio 1:0.5 to 1:2, preferably in the ratio of 1:1, based on the weight.

9. Active-ingredient-containing formulations according to claim 1, comprising one or more pharmaceutical active ingredient(s) selected from the group of the substances from BCS Class I having high solubility and high permeability.

10. Active-ingredient-containing formulations according to claim 1, comprising the active ingredient propranolol and/or pharmaceutically tolerated salts, hydrates or solvate thereof as antihypertensive β-blocker.

11. Active-ingredient-containing formulations according to claim 1, comprising the active ingredient propranolol hydrochloride.

12. Active-ingredient-containing formulations according claim 1, comprising polyvinyl alcohol(s) selected from grades 18-88, 26-88, 40-88 and all grades in between in accordance with the requirements of the Ph. Eur., USP or JPE pharmacopoeias, including grade 28-99 in accordance with the requirements of the JPE or Ph. Eur.

13. Active-ingredient-containing formulations according to claim 1, comprising polyvinyl alcohol(s) selected from grades 18-88, 26-88 and 40-88, in particular from grades 26-88 and 40-88.

14. Active-ingredient-containing formulations according to claim 1, comprising co-mixtures of PVA and microcrystalline celluloses in an amount such that the PVA/MCC content in the final tablet is in the range between 1 to 99% by weight, preferably 5 to 95% by weight, in particular in the range from 10 to 90% by weight, based on the total weight of the tablet.

15. Active-ingredient-containing formulations according to claim 1, as pressed products or compressed tablets having high tablet hardnesses and low friabilities which have been obtained using low compression forces and low injection forces.

16. Directly compressible composition comprising propranolol hydrochloride and a co-mixture consisting of fine-grained PVA and fine-grained MCC which, by compression with a compression force of 20 kN, leads to tablets having hardnesses of greater than/equal to 200 N, which on the other hand have a friability of less than/equal to 0.1% by weight.

17. Directly compressible composition comprising propranolol hydrochloride and a co-mixture consisting of fine-grained PVA and fine-grained MCC which, by compression with a compression force of 10 kN, leads to tablets having hardnesses of greater than/equal to 100 N, which on the other hand have a friability of less than/equal to 0.15% by weight.

18. Tablet produced from a directly compressible composition according to claim 1, comprising propranolol hydrochloride and a co-mixture consisting of fine-grained PVA and fine-grained MCC, which has an extended release of active ingredient of more than 12 hours, where not more than 22% of the active ingredient have been released after one hour, about 25-50% after 3 hours, 50-80% after 6 hours and not less than 80% after 12 hours.

19. Tablet produced from a directly compressible composition according to claim 1 which has extended release of active ingredient, comprising an active ingredient selected from the group of substances from BCS Class I having high solubility and high permeability, and a co-mixture consisting of fine-grained PVA and fine-grained MCC, where the composition comprises:
30-40% by weight of active ingredient,
15-50% by weight of polyvinyl alcohol,
15-50% by weight of microcrystalline cellulose,
0-1% by weight of flow-control agent,
0-1% by weight of lubricant,
and where the total amount of the ingredients adds up to 100% by weight.

20. Tablet according to claim 19, comprising propranolol hydrochloride as active ingredient.

21. Process for the production of tablets according to claim 18, characterised in that finely ground PVA, microcrystalline cellulose and the active ingredient are each sieved in order to remove coarse particles and mixed in the desired amount, and optionally with the weighed-out amounts of the other components, and the mixture obtained is subsequently pressed or compacted to give tablets.
